(19) United States
(12) Patent Application Publication
(10) Pub. No.: US 2026/0363554 A1
(43) Pub. Date: Nov. 19, 2026

(54) PLASMONIC NANOBUBBLE ENDOSCOPE SYSTEM FOR IN VIVO THERANOSTICS OF CANCER CELLS IN TISSUE

(71) Applicant: Scorpido Photonics Inc., San Pedro, CA (US)

(72) Inventor: Dmitri Lapotko, San Pedro, CA (US)

(21) Appl. No.: 19/137,586

(22) PCT Filed: Dec. 11, 2023
(86) PCT No.: PCT/US2023/083308

§ 371 Date: Jun. 10, 2025

Related U.S. Application Data
(60) Provisional application No. 63/432,010, filed on Dec. 12, 2022.

(51) Int. Cl. A61B 5/00; A61K 41/00

(52) U.S. Cl. CPC A61B 5/0084; A61B 5/0062; A61B 5/4848; A61B 5/7264; A61K 41/0052; A61B 2562/0233

(57) ABSTRACT

The invention relates to methods and systems for diagnosis and/or connected diagnosis and treatment (theranostics) of disease (for example, cancer) at the cellular level in vivo through the generation and detection of disease-specific plasmonic nanobubbles (“PNBs”). PNBs are on-demand laser pulse-activated non-stationary vapor nanobubbles. A system for diagnosing and treating the disease in a patient comprises a laser module connected to a flexible probe, which optically generates and detects PNBs. A method for diagnosing the disease comprises (a) administering metal nanoparticles to a patient; (b) navigating a probe in a patient to a target tissue; (c) generating PNBs in vivo with a laser pulse delivered through the probe; (d) detecting PNBs optically in vivo; and (e) diagnosing the disease through analysis of the detected optical signal in response to a laser pulse. The method further comprises treating the disease, based on the diagnostic step, with PNBs or other means.

### BACKGROUND

The present invention relates to methods and systems for diagnosis and/or connected diagnosis and treatment (“theranostics”) of disease (for example, cancer) at the cellular level in vivo through plasmonic nanobubbles (“PNBs”).

When a primary disease, for a example, cancerous tumor or other, is microscopic and in an early stage of having just few cancer cells, it is difficult (if not impossible) to both detect and treat such tumor or other disease. However, when an aggressive tumor grows to a detectable size it is often too late to safely cure it. High-sensitivity single cancer cell in vivo detection at the site of primary disease is not supported by standards of care, while current ex vivo diagnosis (through biopsy) takes a long time, lacks the sensitivity for detecting single cancer cells and does not support any treatment during the diagnostic procedure. Furthermore, current diagnostic and treatment methods are either too invasive (like surgery-based diagnostics) or/and too inefficient, resulting in late-stage treatments with poor outcome and low quality of patient's life.

Intraoperative real-time detection of residual cancer or other target cells in surgical bed with the sensitivity at the level of single cancer cells is currently not supported by standards of care or investigational products. These cancer cells create an MRD (microscopic residual disease) which, in turn, results in local and regional recurrence and metastases, the main courses of complications and deaths for surgically treated cancer patients. Further, standards of care cannot treat unresectable MRD growing along surfaces of critical structures such as nerves or blood vessels.

Despite continuous improvements in onco-surgery, microscopic residual disease (“MRD”) remains a significant problem. Pathological analysis of surgical margins, the main currently available MRD diagnostics, is slow, often inaccurate and not always available. As a result, surgeons routinely resect large margins of normal tissue to remove potential MRD. Unfortunately, this approach often fails, causes high morbidity and reduces patients' quality of life and eligibility. Post-operative radiation or chemoradiation therapies further increase the morbidity, treatment cost and reduce patients' quality of life. Further, MRD often becomes highly resistant to radiation or chemotherapy resulting in poor survival.

Real-time diagnostics and treatment of cancer, including MRD, requires fast execution of three steps: (1) acquisition of the diagnostic data from a tissue with the sensitivity sufficient to detect even single cancer cells, (2) analysis of such data and decision making regarding the next, treatment step and (3) execution of the treatment.

Existing methods and devices do not provide solutions for the above problems and therefore cannot support real-time intraoperative cancer cell diagnostics, treatment and theranostics in a surgical bed or a minimally invasive evaluation of an organ with suspected primary microscopic disease. Moreover, existing diagnostic and treatment paradigms have other shortcomings: (1) pharmaceutical or diagnostic systemic agents remain active regardless whether they reached their target disease or are in normal healthy tissue and thus increase the safety concerns for their use, (2) local treatments such as surgery or radiation therapy cannot efficiently discriminate a target cancer cells from normal tissues thus reducing the treatment efficacy and increasing the damage (morbidity) to normal tissues and cells during the treatment, and/or (3) stationary external energy conversion mechanisms involve high losses of the energy which, in turn, reduces the treatment or diagnostic safety (through collateral damage) and/or the efficacy (by directing only a fraction of the applied external energy into a desired diagnostic or treatment action).

Although new diagnostic technologies are being developed, they still cannot detect MRD in solid tissue in vivo with single cancer cell sensitivity and in real time. As a result, there remains a need for real-time detection and treatment of MRD in vivo.

PNBs are transient vapor nano- or micro-bubbles generated on-demand with a short laser pulse due to heat produced by plasmonic nanoparticles or their clusters upon absorption and conversion of pulsed optical energy. Laser pulse activated PNB explosions with observable mechanical impact at nanoscale can detect single cancer cells, precisely remove tissue at micro-scale, and/or selectively destroy cancer cells without damaging healthy cells. Nature Nanotech 2016 11, 525-532 https://doi.org/10.1038/nnano.2015.343; Nature Med 2014 July; 20 (7): 778-784 doi: 10.1038/nm.3484; J Surg Res 2011 March; 166 (1): e3-e13 doi: 10.1016/j.jss.2010.10.039.

However, PNB generation in tissue in vivo presents logistical problems. Specifically, the delivery of picosecond laser pump pulses with energy sufficient for PNB generation and optical fluence high enough to penetrate into a target tissue (a surgical bed or a surface of interior organ) requires a flexibility and small size of an optical guide and probe only an optical fiber can provide. Rigid optical delivery systems, such as an articulated optical arm (currently employed with medical picosecond lasers), are not flexible and not small enough to be used, for example, with a surgical robot or with a flexible endoscope and for minimally invasive applications in interior organs. The use of a flexible optical fiber for delivery of picosecond laser pulses for PNB generation, however, is restricted by laser-induced damage to the solid glass fibers due to very high optical intensities of picosecond laser pulses required for PNB generation. Alternative use of longer laser pulses, with lower optical intensities and safer for the optical fiber, reduces the efficacy of PNB generation due to the dissipation of the converted optical energy into a tissue, which, in turn, reduces the safety of the procedure due to a thermal collateral damage, and also reduces the sensitivity and selectivity of PNBs for cancer detection and treatment at the cell level. These limitations pose a problem of the efficient delivery of picosecond laser pulse energy into the tissue in a minimally invasive way that is compatible with minimally invasive diagnostic and surgical procedures.

PNB detection in vivo also presents logistical problems because it requires highly specific and sensitive detection in the background of non-PNB effects induced by a laser pulse in non-target tissue. Prior disclosure of PNB detection is of limited translational application because of these logistical problems—disclosed methods for detection of PNBs utilize acoustic detection methods and real tissue comprises a high optoacoustic background that prevents assay sensitivity. For example, acoustic detection of PNBs requires (i) placing relatively bulky acoustic sensors in contact with the tissue and outside of an optical path of a pump laser pulse, a requirement practically impossible to meet under restricted space in in vivo minimally invasive applications, and (ii) distilling PNB acoustic signals [of a single cancer cell] from opto-acoustic background of healthy tissue always also exposed by a pump laser pulse. Such acoustic background significantly reduces the sensitivity and specificity of PNB detection of single cancer cells. No current solution nor design supports a PNB detection with a cancer cell sensitivity in vivo and in a small non-invasive or minimally invasive device.

Therefore, there are needs for: (1) in vivo diagnostics or theranostics that are fast, highly specific, and highly sensitive to detect and destroy individual cancer or other target cells and (2) efficient flexible optical delivery of the pump optical energy to the tissue to generate PNBs in vivo and for the high sensitivity detection of PNBs in vivo. The present invention meets that need by providing for methods and systems for in vivo diagnostics and/or theranostics of cancer or other target cells in tissue through all-optical detection and/or destruction of cancer or other target cells with PNBs, with a flexible delivery and collection of pump and probe optical energies to/from the tissue in a safe, minimally invasive and technically feasible manner.

### SUMMARY OF THE INVENTION

The present disclosure concerns systems, methods, and devices for generation and detection of plasmonic nanobubbles and destruction of same.

In one aspect, the invention concerns a system for diagnosing a tissue in a patient comprising a base module operatively connected to a probe, wherein the probe optically generates and detects plasmonic nanobubbles in vivo.

In another aspect, the invention concerns a method comprising: (a) administering to a patient metal nanoparticle; (b) navigating a probe to a target tissue; (c) generating plasmonic nanobubbles with a pump laser pulse delivered through the probe; (d) detecting plasmonic nanobubbles optically in vivo; and (e) diagnosing the target tissue through analysis of the detected optical signal in response to a pump laser pulse. In an embodiment, the method further comprises treating the target tissue based on the diagnosing step, with plasmonic nanobubbles or other means.

In another aspect, the invention concerns a method for optical generation of plasmonic nanobubbles in tissue comprising: (a) administering to the tissue plasmonic nanoparticles from about 6 hours to about 30 hours before applying a pump laser pulse, wherein the plasmonic nanoparticles support non-stationary plasmon resonance so to allow their efficient clustering by target cells; (b) delivering a pump laser pulse with biologically safe and deep penetrating near-infrared laser wavelength that coincides with a spectral peak of non-stationary plasmon resonance, wherein the pump laser pulse wavelength comprises from about 770 nm to about 790 nm, wherein the pump laser pulse comprises a biologically safe laser fluence in a range 20-100 mJ/cm², wherein the pump laser pulse is above a generation threshold of plasmonic nanobubbles in the tissue, wherein the pump laser pulse comprises a pulse duration from about 5 ps to about 30 ps; (c) inducing a non-stationary plasmon resonance and associated optical absorption, wherein optical energy of the pump laser pulse is absorbed by the non-stationary plasmon resonance; and (d) evaporating the associated optical absorption. In an embodiment, the plasmonic nanoparticles comprise hollow gold particles coated with PEG. In an embodiment, the plasmonic nanoparticles comprise hollow gold particles conjugated with cancer-specific molecules. In an embodiment, the cancer-specific molecules comprise a monoclonal antibody. In an embodiment, the plasmonic nanoparticles are administered at a concentration of about 2-4 mg/kg of body weight. In an embodiment, the plasmonic nanoparticles comprise particles with surface plasmon resonance properties, permanent or non-stationary transient, at the wavelength of a pump laser pulse. In an embodiment, the plasmonic nanoparticles comprise particles capable of developing transient non-stationary plasmon resonance properties during their exposure to a pump laser pulse, such properties absent under exposure of the particles to continuous pump laser beam or a pulsed laser beam with suboptimal duration of a laser pulse. In an embodiment, the plasmonic nanoparticles comprise two or more types of particles capable of targeting cells with different molecular targets, by using more than one targeting vector. In an embodiment, the two or more types of particles comprise several different conjugated antibodies that target different cancer-specific molecular targets. In an embodiment, the plasmonic nanoparticles comprise two or more types of nanoparticles, wherein each type of nanoparticles comprises different plasmonic properties, capable of generating plasmonic nanobubbles while exposed to two or more simultaneous pump laser pulses having different wavelengths that match plasmonic spectra of said nanoparticles. In an embodiment, the step of delivering a pump laser pulse comprises several simultaneous pump laser pulses applied to the tissue at different wavelengths, to pump different particles associated with such wavelengths. In an embodiment, the method further comprises the step of scanning the pump laser pulse within the aperture of an interface optical element, thereby exposing different locations of the tissue in contact with the probe to the pump laser pulse. In an embodiment, the method further comprises the step of delivering consecutive pump laser pulses to the same tissue, thereby improving the diagnostic and/or therapeutic effect of plasmonic nanobubbles.

The present invention also concerns a device for optical generation of plasmonic nanobubbles in tissue comprising: a probe with a flexible optical guide connected to an optical probe, capable of delivering a pump laser pulse, wherein the flexible optical guide is configured to deliver pump and probe laser beams from one or more sources to a probe in contact with tissue without distorting spectral, temporal and energy properties of the laser beams; an interface optical element in the probe capable of providing an exposure of the tissue volume with a pump laser beam; and a tip of the delivery optical fiber capable of being positioned at a distance from about 5 um to about 2 mm from a back surface of the interface optical element, wherein the combination of the distance between the tip and the back surface of the interface optical element, and thickness and refractive properties of the interface optical element forms a diameter of the pump laser beam in tissue in the range from about 0 um to about 400 um from the probe surface, and wherein the direction of the pump laser beam in the tissue along with optical axis of the delivery fiber in the probe is capable of generating plasmonic nanobubbles in front of the probe. In an embodiment, the optical probe comprises a hollow core optical fiber. In an embodiment, the interface optical element in the probe comprises a ball lens. In an embodiment, the device further comprises a diagonal mirror, wherein the interface optical element in the probe comprises a ball lens with a flat in its back, and wherein the interface optical element in the probe directs the pump laser beam into the tissue at an angle from about 45 degrees to about 110 degrees relative to the optical axis of the delivery fiber in the probe by using a combination of the diagonal mirror and the ball lens. In any of the foregoing embodiments, the interface optical element comprises a material with high refractive index and hardness, wherein the interface optical element comprises an optically flat section of its back surface where the pump laser beam enters that optical element, wherein the interface optical element comprises a surface with a spherical shape, and wherein the surface of the interface optical element comprises an anti-reflection coating at the wavelength of a pump laser beam or probe laser beam or both. In an embodiment, the material comprises sapphire. In any of the foregoing embodiments, the device further comprises an optical element between the guide and tissue that modifies the pump laser pulse wavelength, phase, intensity profile, polarization, temporal profile or its divergence or direction, thereby improving the generation of plasmonic nanobubbles in the tissue. In any of the foregoing embodiments, the device further comprises an optical element between the guide and tissue that splits a laser pulse delivered by a flexible guide into two or several laser pulses with similar wavelength, phase, intensity profile, temporal profile or its divergence but these split pulses are being directed into different locations of the tissue, thereby improving the therapeutic and/or diagnostic effect of plasmonic nanobubbles. In any of the foregoing embodiments, the device further comprises an optical element between the guide and tissue that splits a laser pulse delivered by a flexible guide into two or several laser pulses having different wavelength, phase, intensity profile, temporal profile or its divergence or direction, and directs the split laser pulses into the tissue so to improve the therapeutic or diagnostic effect of plasmonic nanobubbles.

The present invention also concerns a method for monitoring the integrity of a delivery optical fiber comprising: delivering a probe laser light to a probe through the delivery optical fiber; collecting the probe laser light which was scattered or reflected by a tissue or by internal parts of the probe, after exiting the delivery fiber, to a photodetector capable of measuring the relative changes in intensity or power of the probe laser light at the wavelength of the probe laser; monitoring the amplitude level of a photodetector signal; and determining damage to the delivery fiber if the level of the photodetector signal irreversibly decreases below a pre-determined threshold.

The present invention also concerns a device for optical detection of plasmonic nanobubbles in tissue comprising: a probe laser beam, a pump laser beam; an interface optical element, wherein the interface optical element collects probe laser light scattered or reflected by plasmonic nanobubbles generated in tissue volume exposed to laser beams; and a flexible optical guide capable of delivering the collected probe laser light to one or more remote photodetectors, wherein the one or more remote photodetectors are capable of generating an electrical output signal specific to a plasmonic nanobubble. In an embodiment, the probe laser beam is capable of being optically separated from the pump laser beam. In an embodiment, the probe laser beam is optically separated from the pump laser beam by using a wavelength different from that of a pump laser, for example, in the range from about 500 nm to about 2000 nm. In an embodiment, the polarization of the probe laser beam optically separates the probe laser beam from the pump laser beam. In an embodiment, the probe laser beam and the pump laser beam are capable of being co-delivered into tissue such that both beams overlap or coincide in the tissue. In any of the foregoing embodiments, the probe laser is capable of delivering a continuous laser beam to the tissue with a very low relative intensity noise at the frequency and time domains associated with the electrical output signal specific to the plasmonic nanobubble. In an embodiment, the very low relative intensity noise at the frequency and time domains associated with the electrical output signal specific to the plasmonic nanobubble comprise a noise level below-150 dB in a time-window up to 10 us, and a frequency range from about 0.02 MHz to about 100 MHz. In any of the foregoing embodiments, the probe laser beam comprises a pulsed beam of the pulse duration from about 1 ps to about 10 ns, and wherein the probe laser beam is time-delayed relative to the pump laser beam by about 20 ns to about 100 ns. In any of the foregoing embodiments, the probe laser beam comprises a diameter, wherein the diameter is limited to minimize the background of the light scattered by the tissue probe laser beam to the level that allows for the optical detection of plasmonic nanobubbles with the same probe laser light. In an embodiment, the diameter comprises from about 10 um to about 400 um. In any of the foregoing embodiments, the probe laser beam is capable of being internally reflected from or scattered by the optical interface surface between the tissue and the probe laser beam such that a scattering of the probe laser light changes when a plasmonic nanobubble is generated in the tissue close to the interface surface, and wherein said changes in the scattering of the probe laser light are capable of being optically detected as a signal associated with the plasmonic nanobubble. In any of the foregoing embodiments, the probe laser beam is capable of being internally scattered by an optical interface surface between the tissue and the probe laser beam, such that a scattering of the probe laser light changes when the probe laser beam comes in optical contact with the tissue, and wherein said changes in the scattered probe laser light are capable of being detected as a signal parameter. In any of the foregoing embodiments, the signal parameter comprises signal base level amplitude associated with the optical contact with target tissue. In any of the foregoing embodiments, the device further comprises a photodetector at the proximal end of the probe that has a single photosensitive element capable of converting a probe laser light intensity, phase, polarization, duration or wavelength into an electrical signal of a time-amplitude or space-amplitude type. In any of the foregoing embodiments, the device further comprises a photodetector at the proximal end of the probe that has multiple photosensitive elements capable to convert a probe laser light intensity, phase, polarization, duration or wavelength into an electrical signal to form a map or an image of the tissue exposed to a probe laser light. In any of the foregoing embodiments, the device further comprises a photodetector at the proximal end of the probe that has optical elements to direct the delivered probe laser light into such photodetector, including a collimating lens, an optical filter at the wavelength of the probe laser or at other specific wavelength, a focusing lens and a photosensitive element of the photodetector at the focus of the focusing lens. In any of the foregoing embodiments, the device further comprises an optical element in the probe capable of delivering the pump and probe laser beams from the fiber guide to tissue without focusing them, i.e. maintaining the desired diameter D in the tissue near the probe, and to collect and collimate the probe laser light scattered by a plasmonic nanobubble generated in the tissue near the probe. In an embodiment, the optical element a ball lens with the radius close to the distance between optical axis of the delivery and collection guides, and with a flat section in its back surface. In any of the foregoing embodiments, the device further comprises a collection guide capable of transmitting the position of plasmonic nanobubble in the tissue, wherein the collection guide comprises many optical fibers with distal tips placed at a specific distance from the back surface of the optical element. In any of the foregoing embodiments, the device further comprises an optical element between the tissue and optical guides capable of being optically transparent for a pump laser beam and for pressure pulses generated by plasmonic nanobubbles, wherein a back surface of the optical element scatters probe laser light back into collection optical guide. In any of the foregoing embodiments, the device further comprises an optical element between the tissue and optical guides capable of being optically transparent for a pump laser beam, wherein a front or back surface of the optical element is capable of internally scattering the probe laser light back into the collection optical guide, wherein the tissue changes the optical properties of the optical element at the probe laser beam wavelength, for example, it flexes, moves or vibrates when plasmonic nanobubble is generated near that surface, and wherein plasmonic nanobubble-induced changes of that surface influence the intensity, power or phase or frequency or polarization of probe laser light that was reflected from or scattered by that surface and was delivered through the collection guide to the photodetector, said influence resulting in plasmonic nanobubble-specific changes of the photodetector output signal. In any of the foregoing embodiments, the device further comprises one or more proximal tips capable of collecting optical fibers, collimating optics, optical filters, and a photosensing element of the photodetector, wherein the one or more proximal tips are integrated into a photonic circuit. In any of the foregoing embodiments, the device further comprises a compact optical probe of the diameter not to exceed 2 mm, wherein the compact optical probe is capable of optically generating and detecting plasmonic nanobubbles in tissue near the tissue-probe interface, wherein the probe comprises a flexible optical conduit with an outer diameter not to exceed 2 mm capable of delivering the pump and probe laser beams from laser sources to the probe, and from the probe to the photodetector(s) and signal hardware and software. In an embodiment, the probe is routed through standard minimally invasive clinical tools, flexible endoscopes (or similar endo-tools like bronchoscope and endomicroscope or else) or catheters. In any of the foregoing embodiments, the device further comprises a compact optical probe of the diameter not to exceed 2 mm, wherein the compact optical probe is capable of optically generating and detecting plasmonic nanobubbles in tissue near the tissue-probe interface, wherein the compact optical probe comprises a free-space optical guide capable of pump and probe laser beams through a rigid guide from laser sources to the probe, and from the probe to the photodetector(s) and signal hardware and software. In an embodiment, the probe is routed through standard rigid endoscopes or other rigid guides. In an embodiment, the rigid guide comprises a rigid endoscope or a needle, capable to introduce the probe to the desired tissue depth, for example, from 0.5 mm to 100 mm through an aspiration needle.

The present invention also concerns a method for optical detection of plasmonic nanobubbles in tissue comprising: illuminating a pump laser-exposed tissue with a probe laser light at a time when a pump laser pulse arrives into the tissue; collecting the probe laser light scattered by the pump-laser exposed tissue to a photodetector capable of measuring the relative temporal changes in the intensity and power of the collected probe laser light; identifying one or more output signal component specific for a plasmonic nanobubble; and detecting a relative change in the intensity of a probe laser light scattered by plasmonic nanobubble. In an embodiment, the one or more output signal component comprises a bell-shaped signal components with a peak, negative or positive, relative to the signal baseline. In an embodiment, the peak is positioned in the time interval from about 5 ns to about 500 ns from the time moment of the application of the pump laser pulse to the tissue. In an embodiment, the one or more output signal component comprises a duration from about 10 ns to about 1 us, measured at the signal amplitude level half of the peak amplitude. In an embodiment, the one or more output signal components are detected from about 5 ns to about 2 us from when the pump laser pulse arrives into the tissue. In an embodiment, the method further comprises providing the device of any of claims 28-32.

The present invention also concerns a method for optical detection of plasmonic nanobubbles in tissue comprising: illuminating a pump laser-exposed tissue with a probe laser light at the time when the pump laser pulse arrives into a tissue; collecting the probe laser light, scattered by the pump-laser exposed tissue, to a photodetector capable of measuring relative changes in a phase, frequency or wavelength of a probe laser light; detecting changes in frequency, phase, or wavelength of the collected probe laser light relative to the frequency, phase, and/or wavelength of the illuminating probe laser light that are specific for an expansion and collapse of plasmonic nanobubble, wherein the changes in frequency comprise a relative increase or decrease of the light frequency by about 20 KHz to about 600 KHz. In an embodiment, the changes in frequency, phase, and/or wavelength are detected from about 5 ns to about 2 us from the time the pump laser pulse arrives to the tissue.

The present invention also concerns a method for optical detection of plasmonic nanobubbles in tissue comprising: contacting a probe laser with the tissue; illuminating the tissue with a probe laser light and a pump laser pulse at the same time; collecting the probe laser light scattered by the probe laser surface to a photodetector capable of measuring relative changes in an intensity, power, phase, frequency or wavelength of the collected probe laser light; and identifying changes in one or more output signals of the photodetector which are specific to plasmonic nanobubbles and their physical effects, wherein the one or more output signals comprise pressure pulses, motion of the boundary of plasmonic nanobubble, or plasmonic nanobubble-induced motion of the tissue. In an embodiment, the one or more output signals are detected from about 5 ns to 2 us from the time moment of the pump laser pulse arrives to the tissue.

The present invention also concerns a method for diagnosing cells with plasmonic nanobubbles, comprising: exposing the cells to one or more pump laser pulses at specific wavelength, duration and fluence in the range from 20 mJ/cm2 to 150 mJ/cm2; exposing the same cells to a probe laser light at the time it receives a pump pulse; collecting and analyzing a probe laser light as an optical signal; deriving quantitative parameters from the signal within the time interval from 5 ns to 2 us after the exposure of the tissue to a pump laser pulse; comparing such quantitative parameters against pre-determined diagnostic thresholds for a target cell type; and determining the presence of the target cell type, wherein the target cell type is present if one or more parameters of the signal of the collected probe laser light match a diagnostic threshold. In an embodiment, the target cell type comprises a cancer cell.

The present invention also concerns a method for selective eradication of a target cells with plasmonic nanobubbles comprising: exposing tissue to 1 to 20 pump laser pulses at a specific wavelength, duration in the range from 10 ps to 50 ps, and fluence in the range from 70 mJ/cm2 to 250 mJ/cm2; delivering pump laser pulses to the tissue through the probe; monitoring a therapeutic effect of each pump laser pulse through optical detection of plasmonic nanobubbles, with quantitative parameters derived for each optical signal; comparing the signal parameters against pre-determined therapeutic thresholds; and adjusting the fluence of next pump laser pulse if the signal parameters did not match the therapeutic thresholds during the previous laser pulse. In an embodiment, the target cells comprise cancer cells. In an embodiment, the method further comprises running an algorithm that compares the quantitative parameters of the probe laser light-detected signals (detected in response to pump laser pulses) to pre-determined thresholds, wherein the comparison concludes whether the cells are disease-positive or -negative (without a human decision being involved), wherein in the case of disease-positive conclusion, the method further comprises additional pump laser pulses of the increased fluence to the same location, while the probe remains in contact with tissue, with the fluence increased to the level in the range from 100 mJ/cm2 to 250 mJ/cm2. In an embodiment, the step of additional pump laser pulses comprises applying a specific number of pump laser pulses, from 1 to 20, within the minimal possible time interval, in the range from 1 ms to 1 s.

The present invention also concerns a method for intraoperative automated detection of residual cancer cells in a surgical cavity comprising: a probe brought in optical contact with the cavity tissue at specific location of a surgical cavity; applying a pump laser pulse and a probe laser light; collecting and analyzing the probe laser light; automatically determining cancer status of a cavity tissue in contact with the probe; and producing the diagnostic data, including the cancer status and the location of the probe. In an embodiment, the method further comprises obtaining diagnostic data for a specific location in the surgical cavity, delivering the diagnostic data to a human capable of making a decision to treat the disease, and performing the next treatment steps based on the diagnostic data. In an embodiment, the method further comprises obtaining diagnostic data for a specific location in surgical cavity, delivering the diagnostic data to a device capable of treating the disease at the specific location. In an embodiment, the method further comprises obtaining diagnostic data for at least two locations in a surgical cavity, wherein the diagnostic data for at least two locations in a surgical cavity is obtained by scanning the probe from a first tissue location to a second tissue location, thereby building a diagnostic map showing the first tissue location and second tissue location in the surgical cavity as a cancer-positive/negative, wherein the scan comprises an optical contact of the probe with the tissue in each location, for example, by pressing the probe into a tissue to a desired pressure, preventing dragging or damaging a tissue during the scan of the probe, for example, by raising the probe until the probe-tissue pressure decreases below specific threshold, before scanning the probe to the next location, and generating a map showing the diagnostic status of all tested locations.

The present invention also concerns a method for detection of cancer cells in a target tissue comprising: providing an endoluminal tool comprising a probe for generation and detection of plasmonic nanobubbles and a flexible optical conduit connecting a probe and base unit with pump and probe lasers and the photodetector; bringing the probe to the target tissue using a standard tool with flexible lumen, for example, a clinical bronchoscope or an endoscope; exposing the target tissue to pump laser pulses; collecting a probe laser light from a target tissue exposed to a pump laser pulse; quantifying the signal of the collected light into specific metrics; and comparing the signal metrics to diagnostic thresholds and determining cancer status, positive or negative of the signal.

The present invention also concerns a method for eradication of cancer cells in a target tissue comprising: detecting cancer cells in a target tissue using the method of the preceding paragraph; exposing the target tissue with a pump laser pulse while the probe remains in optical contact with the diagnosed tissue; applying one to twenty pump laser pulses at specific wavelength, duration from 5 ps to 25 ps, and fluence in the range from 70 mJ/cm2 to 250 mJ/cm2; delivering pump laser pulses to the same tissue location through the probe; monitoring the therapeutic effect of each pump laser pulse through optical detection of plasmonic nanobubbles, with quantitative parameters derived for each optical signal; comparing the signal parameters against pre-determined therapeutic thresholds; and adjusting the fluence of next pump laser pulse if the signal parameters did not match the therapeutic thresholds during the previous laser pulse.

### BRIEF DESCRIPTION OF THE DRAWINGS

FIG. 1 depicts a preferred embodiment of flexible delivery of picosecond laser pulses to the tissue for the diagnostics and treatment of disease in a tissue surface: 1—laser pulse generating and analyzing units, 2—flexible cable to transmit optical and electric energy and liquid, 3—a compact unit that finalizes parameters of a pump laser pulse and directs it into an endoscope 4, this unit is attached to the proximal end of the endoscope and is referred as “proximal unit”; the proximal unit 3 also directs and detects a probe laser radiation to and from an endoscope 4, 4—an endoscope which comes in contact with target tissue 5 and transmits pump and probe laser beam to the tissue and a probe laser light from the tissue.

FIG. 2 depicts a preferred embodiment of a rigid endoscope with a flexible guide: flexible optical guide 2 delivers the pump laser pulse (solid arrow) and a probe laser beam (dashed arrow, 10), shown with the dashed arrow, from the source unit 1 (with probe and pump lasers and signal analysis unit), connected to a flexible optical guide 2, the unit 3 modifies and controls the pump and probe laser beams with the beam compressor/telescope 5, laser beam scanning unit 6 and the endoscope rigid tube 4, photodetector 12 with optical filter 11 (to cut the pump laser light off), to collect the probe laser light 13 backscattered by PNB 8

FIG. 3 depicts a preferred embodiment of a rigid endoscope: pump laser pulse (solid arrow) and a probe laser beam (dashed arrow) are directed through a rigid endoscope in one fixed direction with the beam compressor/telescope 5, diagonal mirror with the hole 17. Elements 5, 11, 12, 17, 18 are the parts of the proximal unit 3 which is mechanically connected to an endoscope tube 4. The pump laser beam 7 and the probe laser beam 10, both of small diameter, are co-delivered to the tissue where they can generate a PNB 8 in case gold nanoparticles are present. Such PNB 8 would scatter the probe laser light back into an endoscope tube 4, and the backscattered light 13 will be collimated by the high-NA lens in the bottom of the endoscope (grey) and directed to the mirror 17, and then to the photodetector 12 through the filter 11 and a focusing lens 18

FIG. 4 depicts a preferred embodiment of a split pump laser with a pump laser head 14 is connected to a pump laser 1 via a flexible optical guide 2. The pump laser head 14 is mechanically connected to a rigid endoscope 4 and includes a probe laser 15. The probe laser beam is mixed with a pump laser beam via the mirror 16. The pump and probe laser beams are directed through a beam compressor/telescope 5, beam mirror or scanner 6, to the tissue where they generate and detect a PNB 8. A PNB scatters a probe laser light 13 back into the endoscope and to a photodetector 12 and through the optical filter 11. Elements 5,6,11,12 are the parts of the proximal unit 3 which is mechanically connected to an endoscope tube 4.

FIG. 5 depicts a preferred embodiment of a system comprising a flexible micro-probe 6 for the generation and detection of PNBs in tissue: 1—pump laser, 2—a coupler of the pump laser pulse into an optical fiber 9 for the delivery of the pump laser pulse, may also include a shutter and an energy attenuator, 3—a probe laser, 4—a coupler of the probe laser beam into an optical fiber 10, may also include a shutter and an power attenuator, may be same fiber as the fiber 9, 5—a photodetector coupled to the optical fiber 11 that delivers the probe laser light collected from PNB(s) 8 which was/were generated in plasmonic nanoparticles-pretreated tissue 12 near the surface of the optical element, a lens 7, with optional flat section in the back and optically-focusing shape or composition towards the tissue, in contact with the tissue 12 and optically coupled to distal tips of optical fibers 9, 10, 11.

FIG. 6 depicts a preferred embodiment for delivery of a pump laser pulse and probe laser beam comprising an integrated micro-probe with bifocal lens elements: 1—probe casing, 2—tissue, 3—an optical fiber for delivery of a pump laser pulse, connected to a pump laser, 4—an optical fiber for the collection of the probe laser light, connected to a photodetector, 5—a negative spherical lens which, in combination with the lens 6, forms an optically flat section of the back surface where the pump and probe beams enter the lenses 5 and 6, 6—a focusing lens with the focus outside and near its outer (bottom) surface, in the tissue 2 adjacent to that surface, 7—PNB in a tissue, near the tissue surface of the focusing lens and within the aperture of the pump laser pulse 8, 9 probe laser beam, 10—PNB-scattered probe laser light which is coupled into optical fibers 4; D1 and D2—diameters of the laser beams at the distal output of the delivery fiber 3 and the lens 6, respectively

FIG. 7 depicts a preferred embodiment for delivery of a pump laser pulse and probe laser beam comprising an integrated micro-probe with the focusing lens: 1—probe casing, 2—tissue, 3—a fiber for delivery of a pump laser pulse, connected to a pump laser, 4—a fiber for the delivery of the probe laser light, connected to a probe laser, 5—a fiber for the collection of PNB-scattered probe laser light, connected to a photodetector, 6—a focusing lens, 7—PNB in a tissue, near the tissue surface of the focusing lens and within the aperture of the pump laser pulse 8, 9—probe laser beam is directed into an aperture (a footprint) of the pump laser pulse in the tissue, 10—PNB-scattered probe laser light

FIG. 8 depicts an embodiment for delivery of a pump laser pulse and probe laser beam comprising an integrated micro-probe with grin (gradient index) lens: 1—probe casing, 2—tissue, 3—an optical fiber for delivery of a pump laser pulse, connected to a pump laser, 4—an optical fiber for the delivery of the probe laser light, connected to a probe laser, 5—an optical fiber for the collection of PNB-scattered probe laser light, connected to a photodetector, 6—a focusing lens, 7—PNB in a tissue, near the tissue surface of the focusing lens and within the aperture of the pump laser pulse 8, 9—probe laser beam is directed into an aperture (a footprint) of the pump laser pulse in the tissue, 10—PNB-scattered probe laser light

FIG. 9 depicts an embodiment with an integrated micro probe with the lens with total internal reflection of the probe laser beam by a PNB: 1—probe casing, 2—tissue, 3—a fiber for delivery of a pump laser pulse, connected to a pump laser, 4—an optical fiber for the delivery of the probe laser light, connected to a probe laser, 5—an optical fiber for the collection of PNB-reflected probe laser light, connected to a photodetector, 6—a focusing lens with facets that direct a probe laser beam at the surface of the lens 6—tissue 2 interface at the angle that is larger than total internal reflection angle in presence of the tissue or liquid, but smaller than the total internal reflection angle when a vapor is present at the lens surface, 7—PNB in a tissue, near the tissue surface of the focusing lens and within the aperture of the pump laser pulse 8, 9—probe laser beam is directed into an aperture (a footprint) of the pump laser pulse in the tissue, 10—probe laser light after the total internal reflection from the area of the tissue surface of the lens where PNB has created a vapor at the lens surface.

FIG. 10 is a flowchart illustrating the method of the present disclosure.

FIG. 11 depicts an embodiment of a hollow core optical fiber (1) used in a micro-probe (8) to deliver to the tissue (5) a probe (dashed arrow) and pump (solid arrow) laser beams through a lens of a ball type with a flat back section, such flat section preventing the focusing of laser beams (which exit the fiber (1)) at a lens 4—tissue 5 interface surface and forming required diameters of the laser beams at that interface surface; the lens 4 is separated from the tip of the fiber (1) by the gap h, 3 is one or several collecting optical fibers, with their tips shifted closer to the lens relative to the tip of the fiber 1, fibers (3) being made of fused silica or other solid type, to collect a PNB-scattered probe light and to deliver such light to the photodetector at proximal end of such optical fibers.

FIG. 12 depicts an embodiment of a flexible endoscopic micro-probe connected to a laser base unit with a flexible optical fiber bundle, bifurcated on its proximal end, into two legs, each connected to a fiber coupling unit in the base laser unit: the delivery hollow core fiber 1 is coupled to a pump and probe laser beams, the collection optical fibers 3 are optically coupled to the photodetector which receives a PNB (2)—backscattered probe laser light through the collection fibers 3. Distal ends of delivery fiber 1 and collection fibers 3 are bundled into a casing 8 and deliver the probe and pump laser beams to the tissue 5 through the lens 4 which has a flat surface in its back and is separated from the tip of the delivery fiber 1 by the gap h, and directs the pump and probe laser beams into the tissue 5, and collects the probe laser light back-scattered by PNB 2 and direct such light into collection fibers 3.

FIG. 13A and FIG. 13B depict A delivery guide (1) delivers pump (4) and probe (5) laser beams to the tissue (3) through a flexible optical element (2), such optical element is designed to deliver the pump laser beam (4) from the fiber guide (1) to tissue (3), to reflect or scatter the probe laser beam (5) by its back or front surface, and to flex in response to the generation of plasmonic nanobubble (8) so that the portion of the reflected or scattered probe laser light (6) is directed to the collection guide (7) (A) so the probe laser light delivered by the collection optical guide (7) is caused by a PNB 8 which, in turn, causes the flexing of the optical element 2. Figure A shows a PNB-induced increase in the photodetector signal, relative to its base level in the absence of a PNB, due to the increase of the scattered/reflected probe laser light (6) through the optical collection guide (7). Figure B shows a PNB-induced decrease in the photodetector signal, relative to its base level in the absence of a PNB, as detected the scattered/reflected probe laser light (6) through the delivery guide (1). In this case, the PNB 8—backscattered light 6 is collected and delivered to the photodetector by the delivery optical fiber 1

### DETAILED DESCRIPTION OF THE INVENTION

The present invention is directed to systems and methods for all-optical generation and detection of PNBs in cancer or other target cells or tumors in vivo. The optical back-scattering of light by PNBs generated in vivo provides for specific minimally invasive detection in vivo. A minimally invasive optical probe for PNB generation and detection in vivo comprises a novel design of optical fibers and lens to achieve flexible delivery and collection of optical energy between its sources and detectors, on one end, and the target tissue, on another end, and high-sensitivity detection of cancer- or other disease-specific PNBs in vivo. Moreover, the optical probe provides a local and low energy pump laser pulse to safely generate PNBs in vivo to minimize non-specific laser-induced laser damage to healthy tissues. Moreover, systems and methods support dual theranostic functions of PNBs in vivo of detecting (through optical scattering) and treating (through an instant mechanical impact) the disease in vivo in one instant connected procedure. The diagnostic and treatment functions of PNBs can be used in vivo together or separately.

A fundamental diagnostic advantage of a PNB mechanism is in how it employs external energies, compared to other diagnostic methods which also use external energies: To detect cancer cells (or other target) in tissue, an external energy is applied to cancer target cell-bound agents. These agents convert external energy into a detectable signal energy through two mechanisms: absorption and scattering (reflection). A target signal results from the amount of absorbed pump energy and the absorbing volume (number of targets) less the background signal generated by non-target tissue volume (for example, by healthy tissue). Naturally, the smallest target signal is associated with a single target cell. Therefore, to detect a single target cell in a non-specific background, the absorbed energy should be high enough to produce a detectable signal above the background signal. However, in all absorption methods, including fluorescence, the agent damage threshold limits the maximum absorbed energy such agent can emit and hence the detectable signal from a target. Coupled with a small volume of a single target cell and the high volume of the background, this creates a fundamental detection and diagnostic limit for all absorption methods, including fluorescence: they cannot detect targets, a tumor, for example, smaller than millimeters, and makes the detection of single target cells in vivo in the tissue practically impossible. To further increase a target signal, the external energy must be scattered or reflected by the agent instead, without damaging the agent.

Optical scattering by PNB is very high, 10-1000 times higher than that of gold nanoparticles or any other particles, with their vapor-condensed matter boundary reflecting or scattering the light. This boundary cannot be damaged by a probing energy which is reflected or scattered by that boundary. Therefore, the probing optical energy can be increased to the level sufficient to detect a single target cell through the optical scattering or back-scattering of that probing energy by a PNB.

PNBs are generated on-demand in target cell with the 2nd, pump laser pulsed beam of low energy, using plasmonic nanoparticles. A non-stationary transient nature of a PNB requires a small amount of the optical pump energy to create a PNB, compared to much higher optical energy required to create any stationary diagnostic effect, like fluorescence, photothermal or photoacoustic. This makes PNBs very safe compared to other diagnostic methods. Literally, a PNB does not exist in a patient before or shortly after a pump laser pulse.

The unique biological-physical mechanism of PNB generation through nanoparticle clustering-optical energy threshold generation creates a high target cell selectivity of PNBs compared to that of nanoparticle targeting.

Therefore, PNBs detect a target cell by using two types of external optical energy, a pump (to create a PNB through plasmonic absorption) and a probe (to detect it optically through optical scattering). This results in a high target signal to background ratio sufficient to detect a single target cell in tissue in non-invasive or minimally invasive way, for example, by using a low safe pump energy to generate a PNB, and using a high energy at different and safe wavelength to optically detect a PNB.

Previous disclosures of PNB diagnostics and/or theranostics are distinguished from the present invention as follows:
a. Lapotko, Oraevsky 2005 US64601805P, U.S. 60/646,018 [5] discloses no diagnostic and theranostic functions, no designs like a fiber optical probe.
b. Lapotko, Zharov 2007, U.S. Pat. No. 7,230,708 [6] discloses no diagnostic and theranostic functions in tissue in vivo, no designs like a fiber optical probe for PNB optical generation and detection in tissue.
c. Lapotko et al 2013 provisional 61/720,135 [7] discloses no designs like a fiber optical probe for PNB generation and optical detection in tissue. Instead, it discloses preferably acoustic detection of PNBs in tissue. This reference mentions a “fiber optic delivery” of optical energy to tissue without explaining how picosecond optical pulses can be delivered through an optical fiber without damaging it and without distorting parameters of a laser pulse.
d. Publications by Lapotko [8,9] describe optical generation and detection of PNB in vivo in a very unique case of a transparent animal, a zebrafish embrio, which allowed using an optical microscope, with a microscopic in vivo model, an optically-transparent zebrafish embryo, and a probe laser beam through the embryo, being detected behind the tissue. In real tissues in vivo, it is impossible to practically realize the described case and methodology because it is impossible to direct a probe laser beam through the whole tissue, and to detect it after it passed through such tissue, due to a strong optical attenuation and due to anatomical restrictions in vivo, hence our invention uses a backscattering of the probe laser light by PNB at and near the target tissue surface, in combination with a small fiber optical probe which has not been disclosed in [8,9].

Moreover, other diagnostics, summarized in the table below, cannot instantly detect cancers or other target diseases in vivo with a single target cell sensitivity nor can they destroy the detected cells in one connected real-time procedures, the functions our invention supports through the fiber optical probe design to optically generate and detect PNBs in target cells in vivo in tissue:

| | Laser | | | | | |
|---|---|---|---|---|---|---|
| | endo- | | Opto- | | Radioactive | |
| Technology | microscopy | Fluorescent | acoustic | Ultrasound | probes | PNB |
| Detects | no | no | no | no | no | yes |
| single | | | | | | |
| cancer cells | | | | | | |
| in vivo | | | | | | |
| Destroys | no | no | no | no | no | yes |
| detected | | | | | | |
| cancer cells | | | | | | |
| in one | | | | | | |
| theranostic | | | | | | |
| procedure | | | | | | |
| In vivo | None | Fluorescent | Dyes or gold | Optional, | Radioisotopic, | Low dose |
| contrast | | dyes, toxic | nanoparticles | microbubbles | toxic | gold |
| agents | | | | | | nanoparticles |
| Reference | Clinical | Explorer | Opto-acoustic | Probes for | Gamma | [1-3, 8-12] |
| | product, | Air by | tomography, | robotic | probe, Sensei, | |
| | CellVisio | SurgVision | ItheraMedical, | surgery [16] | for radio- | |
| | [19], | [14] | not in | and | guided | |
| | clinical | Quest | clinic[13] | bronchoscopy | surgery [17] | |
| | results [20] | Spectrum | | [18] | | |
| | | [15] | | | | |

The references to the foregoing paragraphs regarding previous disclosures are as follows:
1. Nature Nanotech 2016 11, 525-532 https://doi.org/10.1038/nnano.2015.343
2. Nature Med 2014 July; 20 (7): 778-784 doi: 10.1038/nm.3484
3. J Surg Res 2011 March; 166 (1): e3-e13 doi: 10.1016/j.jss.2010.10.039
4. Lapotko patent U.S. Pat. No. 10,471,159B1-Diagnosis, removal, or mechanical damaging of tumor using plasmonic nanobubbles-Google Patents
5. Lapotko patent WO2006078987A2-Laser activated nanothermolysis of cells-Google Patents
6. Lapotko patent U.S. Pat. No. 7,230,708B2-Method and device for photothermal examination of microinhomogeneities-Google Patents
7. Lapotko patent app US Patent Application for MULTIFUNCTIONAL CHEMO-AND MECHANICAL THERAPEUTICS Patent Application (Application #20140120167 issued May 1, 2014)—Justia Patents Search
8. D S Wagner, N A Delk, E Y Lukianova-Hleb, J H Hafner, M C Farach-Carson 2010 Biomaterials 31 (29), 7567-7574
9. E Y Lukianova-Hleb, C Santiago, D S Wagner, J H Hafner, D O Lapotko 2010 Nanotechnology 21 (22), 225102
10. D Lapotko Cancers 2010 3 (1), 802-840
11. D. Lapotko Cell theranostics with plasmonic nanobubbles, in: Applications of Nanoscience in Photomedicine 2015 February 3, Elsevier, pp 105-130 https://doi.org/10.1016/C2013-0-23222-0
12. Provisional patent application 62/010,981 filed Jun. 11, 2014
13. Multi-spectral optoacoustic tomography by iThera Medical https://ithera-medical.com/products/
14. Intraoperative fluorescent imaging system by SurgVision https://www.surgvision.com/
15. Quest Spectrum fluorescent imaging intraoperative system https://www.quest-mi.com/products.html
16. Ultrasound intraoperative system https://www.bkmedical.com/systems/bk5000-robotic-surgery-ultrasound-system/
17. Gamma probe for radio-guided surgery https://senseisurgical.com/
18. Radial EBUS probes for lesion location confirmation Radial EBUS Probes|Olympus America|Medical
19. Laser Endomicroscopy, a clinical product, CellVizio: https://www.maunakeatech.com/en/physicians/11-cellvizio-system
20. Clinical study with CellVisio and robotic surgery: https://pubmed.ncbi.nlm.nih.gov/26626214/

The system disclosed herein comprises base module (1) with lasers and signal electronics and software, operatively connected (2) to a probe (3), (4) which optically generates and detects PNBs in plasmonic nanoparticles-pretreated tissue. The probe of the present invention provides for high-sensitivity, real-time detection of cancer cells in vivo in tissue or organ surface to the depth of 0 to 1000 um; and precise targeted nano-surgery, with mechanical immediate selective on-demand destruction of target cells, with the tissue depth from 0 to 1000 um.

The probe has two design options, with microscopic tip connected to the fiber bundle or with a rigid endoscope-type probe

In an embodiment, the probe comprises a rigid endoscope optimized for robotic surgery. In another embodiment, the probe comprises a flexible micro-probe optimized for flexible endoscopy or robotic surgery. Both of the foregoing embodiments comprise a flexible optical connection between the base module and the probe.

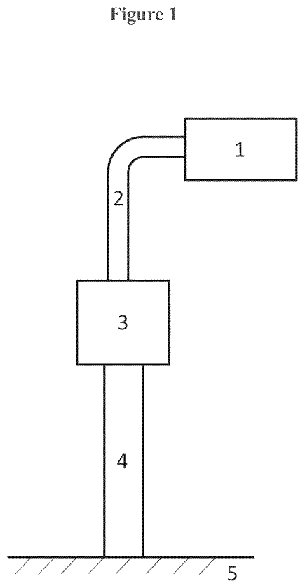

A preferred embodiment of the rigid endoscope disclosed herein is depicted in FIG. 1. The rigid endoscope is compatible with robotic surgery to be inserted through a port. An advantage of a rigid endoscope is an ability to intraoperatively detect and destroy cancer cells at a specific location in the tissue. The rigid endoscope can scan the tissue area within its footprint and can immediately access the target tissue area through a standard surgical robotic port. In an embodiment, the rigid endoscope diameter comprises from 5 to 12 mm and the length from 100 to 500 mm.

In an embodiment, “rigid endoscope embodiment A,” the pump laser pulse from a pump laser (1) is delivered to a rigid endoscope unit (3) via an optical fiber or fibers (2). In the unit (3), the pulse is passively modified to increase its fluence to the level of PNB generation in endoscope (4). The unit (3) supports the pointing or scanning of pump laser pulse over the tissue and the optical detection of PNBs generated in the tissue.

Rigid endoscope embodiment A provides for flexible delivery of a pump laser pulse which is fully formed in unit 1. Generation of PNBs in tissue requires high optical intensity, which could potentially damage optical fiber. A safe flexible delivery of the pump laser pulse and the PNB generation with that pulse are realized through several solutions.

In an embodiment, the rigid endoscope embodiment A comprises a single optical fiber of large core diameter, for example, 200 um to 1000 um, to deliver the pump pulse. A pump beam is expanded before being launched into the fiber so its intensity at the launch fiber-air interface is below the fiber laser damage threshold. This can be achieved by using, in addition, a fiber window cap or a taper to further increase the aperture of the pump beam and thus to further reduce the optical intensity to a low safe for the fiber level. In an embodiment, to further prevent the damage to the optical fiber-air interface, the surface of the fiber window can be processed to reduce the possibility of the damage, for example, by (1) polishing the core fiber tip or/and (2) by creating special nano-pattern, like a “motheye” [see, for example: https://www.molex.com/molex/products/family/rare_motheye_fiber?parentKey=fiberguide] at that surface of the fiber tip. In an embodiment, to further prevent the damage to the fiber-air interface, the air volume between the coupling lens, the lens that couples a free space pump laser beam into the fiber, and the fiber tip, is enclosed to protect this volume from dust. In an embodiment, to further prevent the damage to the fiber-air interface, the air volume between the coupling lens, the lens that couples a free space pump laser beam into the fiber, and the fiber tip, is enclosed and filled with other than air dust-free gas, for example, nitrogen, to reduce the probability of the damage, or with vacuum.

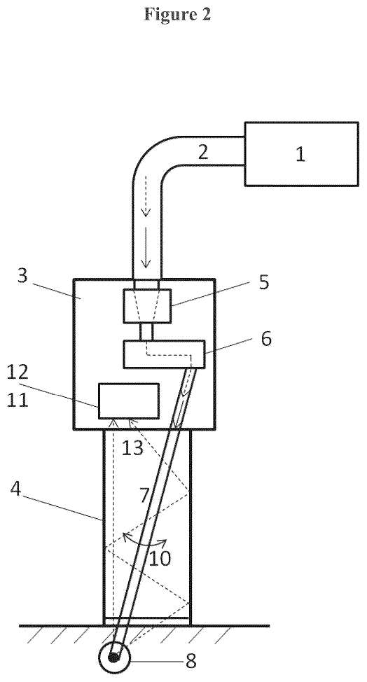

In another embodiment, the rigid endoscope embodiment A comprises a fiber bundle, for example a bundle of multipole fibers, each fiber in the bundle having a small core. In this embodiment, the pump laser pulse is split into many pulses of low energy and low intensity (1), safe to launch into standard fused silica multimode optical fiber. Several fibers deliver the total pump pulse energy through a flexible fiber bundle (2), with the length of 2 m, for example, which is short enough to prevent severe temporal and spectral distortion of the laser pulse, to a scan unit (6), where the endoscope is attached to a flexible fiber bundle. FIG. 2 depicts a preferred embodiment of the rigid endoscope comprising a fiber bundle.

To avoid a significant increase of the duration of the pump laser pulse due to optical dispersion inside optical fiber of the rigid endoscope embodiment A, and thus to avoid a decrease in the energy efficacy of PNB generation, a pump laser beam is launched into the fiber to provide a low numerical aperture of the laser beam inside such optical fiber and at the exit from the fiber, for example, in the range 0.02-0.07. Such low numerical aperture of the laser beam can be realized in the fiber with much higher numerical aperture, for example, 0.1 to 0.22. In an embodiment, this is achieved by launching a divergent laser beam into the fiber, with the laser beam entering the fiber surface beyond the focal waist of the beam, and the beam aperture at the entrance being smaller than the core aperture of the fiber, for example, 50% to 90% of the core aperture. The focal length of the coupling lens should be long enough, for example, from 15 mm to 75 mm, for a fiber with the core 30-300 um. As a result, the laser beam propagates inside the fiber core close to its axis and this propagation mode reduces both the broadening of the pulse duration and the probability of the laser damage to the fiber inside its core.

In an embodiment, instead of solid core optical fiber, a fiber with a hollow core structure engineered to guide a laser pulse can be used. This can be, for example, a hollow core photonic crystal fiber or a hollow core anti-resonant optical fiber. Such fibers transmit very short laser pulses of high energies without the damage to the fiber and without distortion of temporal and spectral properties of a laser pulse

In an embodiment, the rigid endoscope embodiment A also comprises an increased optical fluence through compression of the pump laser beam. To prevent a pump laser beam of low fluence, insufficient for PNB generation in the tissue, the pump beam is compressed to a smaller diameter, increasing the optical fluence of the pump laser pulse to the level sufficient for the generation of diagnostic and therapeutic or surgical PNBs in cancer cells in a tissue. In the embodiment comprising a fiber bundle for the delivery of a pump laser beam, the device may include an additional optical homogenizer (3) to create a uniform laser beam (4), without hot spots, out of the fiber bundle. The pump beam may have a relatively large diameter and low fluence. This beam is compressed or partially focused with the telescope or similar lens combination (5) to the smaller diameter (for example, from 20 to 200 um) at the endoscope-tissue interface, with high enough fluence to generate PNBs in cancer cells in a target tissue.

In an embodiment, the rigid endoscope embodiment A allows for directing the compressed pump laser beam of small diameter into the tissue surface through an endoscope. The pump laser beam may have a permanent fixed direction in the endoscope. The mirrors or other elements (6) as depicted in FIG. 2 point the pump and probe laser beams into specific fixed position at the endoscope output window, for example, in the center of an endoscope footprint. In this case, there is one specific location of PNB generation in tissue surface, relative to the distal end of an endoscope, for example, its center, and the distal diameter of an endoscope can be reduced (a tapered endoscope) in order to provide a better access to small targets and in a restricted space. In this embodiment, the pump laser beam is scanned within an endoscope footprint at the distal end. The compressed beams are directed into a scanner (6), and this scanner directs and scans the pump and probe beams (7) and (10), respectively, across the footprint of the endoscope. The scan speed and the pump pulse repetition rate are synchronized to provide a full exposure of the tissue area covered by an endoscope by the pump laser pulses at the distal end of the endoscope, within the footprint of an endoscope. PNBs (8) are generated at the tissue surface within the aperture of the pump laser beam (7) and within the footprint of an endoscope. In some embodiments, to better access specific locations, the tissue interface window of the endoscope can be tilted at specific angle, for example, 45 or 90 degrees, with pump and probe laser beams delivered to the tissue surface and the PNB-backscattered probe laser light collected from there at that angle. In any of the foregoing embodiments, the tissue depth of PNB generation is controlled via the fluence of the pump laser pulses, from the surface only PNBs at the minimal pump laser fluence, and the maximal depth at the highest pump laser fluence.

In an embodiment, the rigid endoscope embodiment A also provides delivery of a probe laser beam: an additional c.w. (continuous wave) probe laser beam (1′) is delivered to and detected as a backscattered by PNB probe light from the tissue surface. The delivery of the probe laser beam uses the same optical path as that for the pump beam. The probe beam is optically mixed with the pump beam before being launched into a fiber bundle. The probe laser beam (10) is co-delivered to the same location in tissue as the pump beam, through the fiber bundle, homogenizer, compressor, fast scanning mirror and the endoscope window at the distal end.

In an embodiment, detection of backscattered light generated by PNBs exposed to probe laser light provides for the detection of cancer cell-generated PNBs. The backscattered light (FIG. 2) that reaches the proximal end of the endoscope may be filtered out with an optical filter (11), and can be detected by a photodetector (12) positioned behind the optical filter, both the filter and photodetector may have a circular shape with the hole in the center to allow through the scanned laser beams 7 and 10. The inner surface of an endoscope would be made optically-reflecting for the probe light (13) to deliver more backscattered light to the photodetector and thus to improve the diagnostic sensitivity.

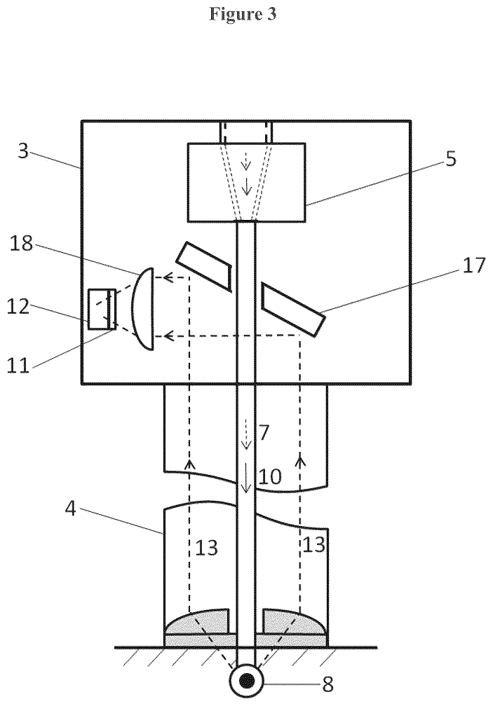

In an embodiment, to improve the diagnostic sensitivity, the backscattered light is detected close to a distal window of the endoscope, where the amount of backscattered light is the maximal. This would have the photodetector and optical filter installed in the distal tip, close to the endoscope window and around the scanned area. Alternatively, or in addition, another embodiment as depicted in FIG. 3 improves the diagnostic sensitivity by collecting more PNB-backscattered probe laser light by adding two optical elements into a design with the fixed laser beam. A high numerical aperture lens at the output window of the endoscope collects and collimates the PNB-backscattered probe laser light back into the endoscope, and the diagonal mirror (17) which directs the backscattered probe laser light through the lens (18) to the photodetector (12), with an optional optical filter (11). Both elements, the collimating lens and diagonal mirror, have holes to allow for the delivery of the pump and probe laser beams (7) and (10), respectively, from their sources and to the endoscope window.

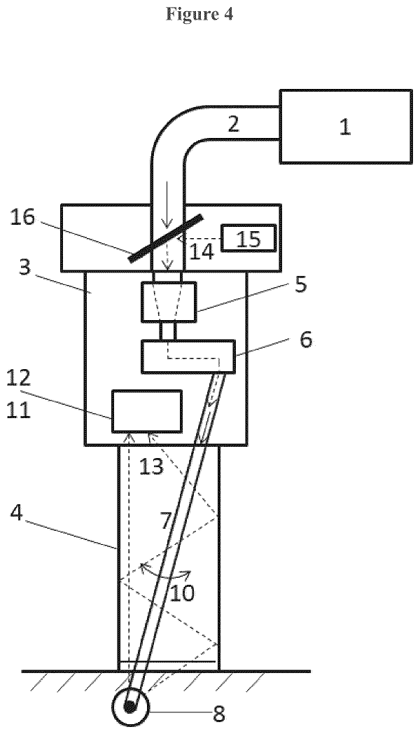

In another embodiment depicted in FIG. 4, “rigid endoscope embodiment B,” the pump laser pulse is generated in the rigid endoscope unit (3) which incorporates a laser head, a part of the pump laser. In this solution, a pump laser is split into two components, a base unit (1) and a head (in the unit 3) connected via a flexible cable (2).

FIG. 4 depicts a preferred embodiment of rigid endoscope embodiment B. In this preferred embodiment, the final parameters of a pump laser pulse are achieved in a compact component (14) of the pump laser (a pump laser head) which is connected to the rest of the pump laser with a flexible optical/electrical/liquid cable (2) and has a shape and dimensions allowing its direct use with a surgical endoscope or robotic arm or port. The unit (14) forms the final picosecond pump pulse and also mixes it with a probe laser beam which is delivered from a probe laser (15) through a mixing mirror (16). The unit (14) is permanently attached to the endoscope through the unit (3) (FIG. 4) and thus is mechanically connected to a proximal end of an endoscope tube (4). For PNB applications, a unit (14) may be a picosecond laser head, compact enough to fit within a proximal end of an endoscope. The laser pulse length should be in the range 5-40 ps to minimize the laser energy dose to the tissue and to prevent laser or thermal damage to the tissue.

The combination of the pump laser head attached to a rigid endoscope provides precise control of the parameters of the laser pulse (e.g., wavelength, duration, and energy and fluence), which is critical for PNB generation. Moreover, this combination prevents detrimental non-linear optical effects associated with the propagation of picosecond laser pulse in a fused silica optical fiber (for example, temporal and spectral broadening). Alternatively, such detrimental effects can be prevented by using a hollow core optical fiber as described above.

In an embodiment, an endoscope tube (4) may, for example, be 400 mm long and outer diameter 8 mm or 6 mm. An aperture exposed to a pump laser pulse, fixed or scanned within a distal end of an endoscope, may have a diameter up to 5 mm. If the photodetector (12) is installed in the distal part of an endoscope, the laser scanned aperture of the tissue may be reduced to 1-4 mm.

In an embodiment, to better access specific locations, the distal window is tilted at, for example, at 90 degrees, with pump and probe laser beams delivered to the tissue surface and the PNB-backscattered probe laser light collected from there at that angle (when target tissue is on a side of an endoscope). In this case, an additional mirror or a prism can be employed to re-direct optical beams.

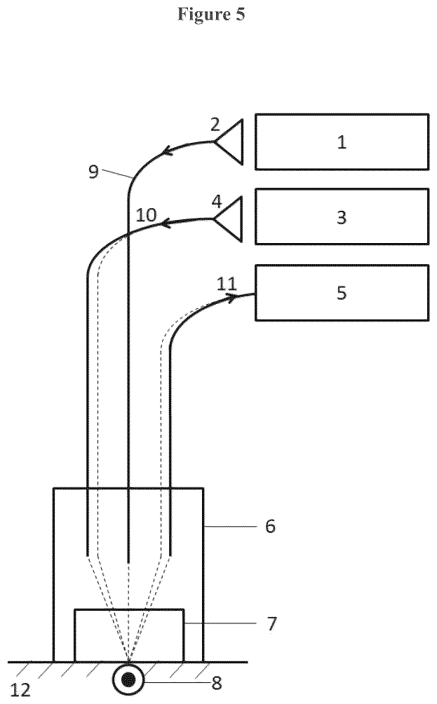

A preferred embodiment of the flexible PNB micro-probe disclosed herein is depicted in FIG. 5. A high flexibility (with bend radius similar to that of optical fiber, i.e. down to 10 mm) and a small size (down to 0.5 mm of the probe diameter) of the flexible micro-probe allow its precise and unrestricted minimally invasive manipulation in a patient, in order to access any target under a manual or robotic control of the micro-probe with endoscopic or surgical or biopsy devices. In an embodiment, the flexible micro-probe diameter comprises from 0.3 mm to 3 mm and the length from 3 mm to 30 mm.

With a flexible PNB micro-probe, the laser beams cannot be scanned inside the probe, as in a rigid PNB endoscope. Therefore, different locations of the target tissue can be accessed by scanning an entire micro-probe across the surface of the target tissue.

An embodiment of the system incorporating a flexible PNB micro-probe is depicted in FIG. 5. The flexible micro-probe comprises a picosecond pulsed pump laser 1 with the laser beam modified by an optical fiber coupling unit 2 to safely launch the maximal possible pulse energy into the optical fiber 9. The flexible micro-probe further comprises a low amplitude/intensity noise continuous probe laser 3 beam modified by an optical fiber coupling unit 4 to launch it into an optical fiber 10, wherein optical fiber 10 and 9 optionally can be the same fiber. The flexible micro-probe further comprises one of several optical fibers 11 to collect and deliver the PNB-scattered probe beam light to the photodetector 5 which converts the PNB-scattered light into an electrical signal with parameters specific to a PNB.

In an embodiment, the flexible micro-probe 6 comprises distal ends of optical fibers 9, 10, 11 and has an optical element 7 to optimally direct the pump and probe laser beams into the tissue surface 12 to generate and optically detect a PNB 8 in the tissue, and to collect and direct back into the optical fiber(s) 11 the probe laser light scattered or backscattered by PNB 8. The outer surface of optical element 7 is in optical contact with target tissue 12 and acts as an optical interface for the laser beams directed to and collected from the tissue surface.

In an embodiment, a pump laser pulse is delivered into a tissue surface by the flexible micro-probe. The delivery of the pump laser pulse via the optical fiber (9) (one or several) at the low fluence, with the expanded beam and large core diameter of the optical fiber (from 200 um to 600 um), to avoid the laser damage to the fiber, and the concentration of the pump laser beam after it leaves the fiber tip and before it arrives to the tissue, with an additional optical element, a lens, installed between the distal tip of the optical fiber and the tissue (as depicted in FIG. 5). Alternatively, a fiber (9) can be a hollow core optical fiber with the core diameter from 10 um to 100 um, and engineered to deliver the pump and probe laser beams from the lasers to the tissue without laser-induced optical damage and without distorting the pump laser pulse, and with the minimal energy loses of the pump laser pulse energy

In an embodiment, the pump and probe laser beams have their diameters at the tissue surface within specific range, for example 40-50 um. This range may vary from 20 um to 1000 um.

In an embodiment, the delivery of a pump laser beam out of optical fiber includes a concentration of the pump beam by the lens (7) to increase its optical fluence at the tissue to the desired level sufficient to generate PNBs. For example, for 300 um core fiber, the beam diameter at the lens output surface will be 30-150 um, thus increasing the fluence by 4-100 times (compared to a low and safe fluence level in the fiber), to the level sufficient to generate PNBs in tissue near the lens outer surface. Such lens is positioned at a specific distance from a fiber tip and may have more than one optical focus. To generate PNBs in a larger (than the diameter of a pump laser beam) area of the tissue, the probe is mechanically scanned across the tissue surface by, for example, a flexible or surgical robot. Below there are several design options for the delivery of the pump laser pulse:

Delivery of the pump laser pulse may be accomplished a number of ways.

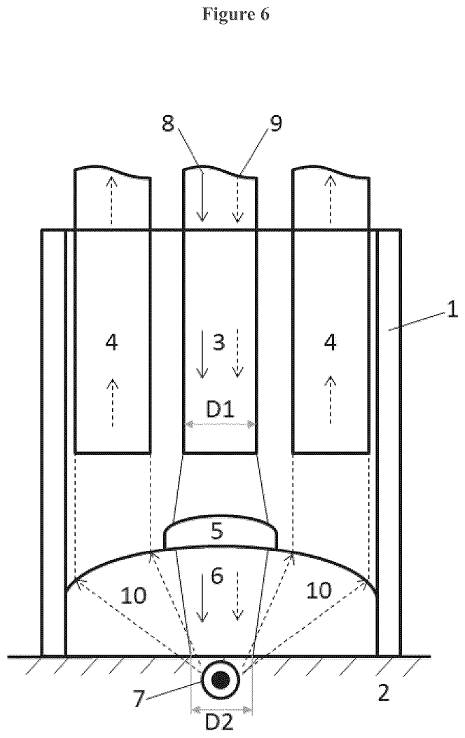

In an embodiment, depicted in FIG. 6 an integrated micro-probe includes one central “delivery” fiber (3), this central fiber delivers both the pump beam (8) and probe laser beam (9) to the skin surface (2) through the two optical elements, negative lens (5) of small diameter, close to that of the fiber core, and positive lens (6) which concentrates or expands the beams from the fiber core diameter D1 to a diameter D2 at the tissue surface (2) so that D1/D2=0.3-3, without fully focusing pump and probe beam at the tissue surface. The main focus of the optical lens (6) is in the surface of the tissue (at the tissue depth range from 0 um to 200 um), so that any probe laser light scattered by the PNB (7) in the tissue is collimated by the lens (6) and then is coupled into collection optical fibers (4) with large NA and core which collect the maximal amount of PNB-backscattered probe laser light. In this design, an optical element (6) between the fibers and tissue is bifocal: a longer focal distance for the central area where the pump and probe beams are delivered to the tissue as collimated beams, and a shorter focal distance, matched to the tissue surface, for the peripheral area of the optical element (a lens (6)). In this case, this is achieved with the combination of two lenses 5 and 6 but, also, can be achieved with a single bi-focal lens (6). The probe diameter and length are minimized in order to support its maximal access to targets in restricted locations (for example, to lung nodules via airways, through lumens of endoscopes or through biopsy needles).

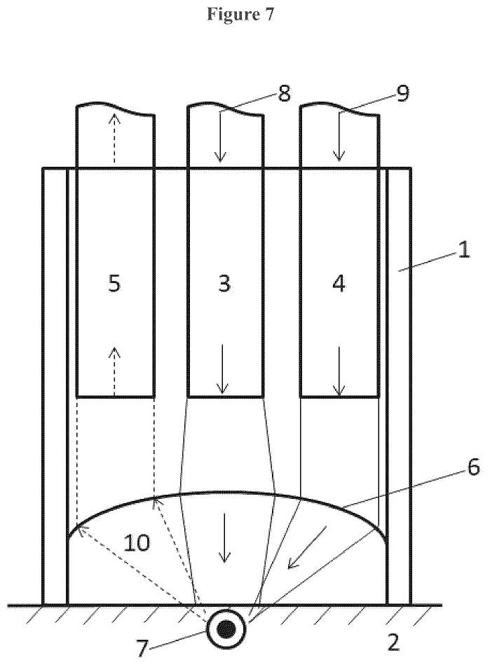

In another embodiment, depicted in FIG. 7, an optical element in the probe, between the optical fibers and the tissue, is a focusing lens separated by air from the fiber tip. The lens partially focuses the pump beam so its diameter at the output surface of the lens (the one in contact with tissue) is for example, from 20 um to 200 um. The focal point of the lens is located well outside the lens and on the tissue side. The fiber side of the lens may have a convex or a combination shape. The tissue side of the lens can be flat or convex. The probe diameter is from 0.5 mm to 3 mm.

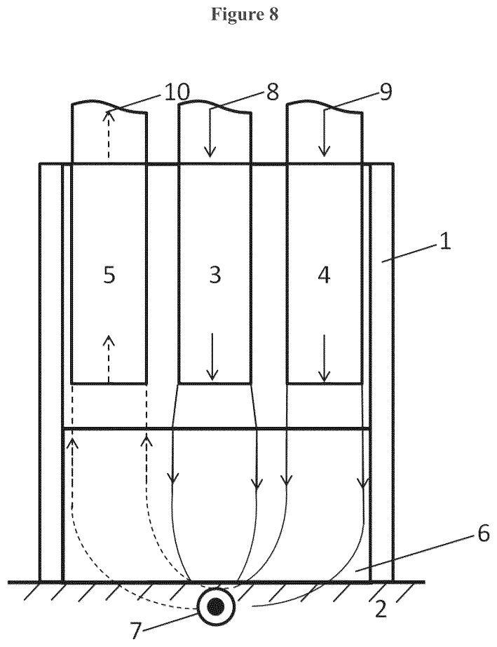

In another embodiment, depicted in FIG. 8, an optical element 6 in the probe, between the optical fiber and the tissue is a grin (gradient index) lens (FIG. 8) with two flat parallel fiber and tissue surfaces. Such grin lens partially focuses the pump beam to reduce its diameter on its tissue surface while the focal point of such lens is outside the lens and on a tissue side. This can be a grin lens with a pitch between 0.2 and 0.24, and the diameter between 0.5 mm and 2.0 mm.

In any of the foregoing embodiments, the delivery of a picosecond pump laser pulse through the optical fiber is optimized to minimize a temporal broadening of the pump laser pulse in the optical fiber, and so to avoid an associated decrease in the PNB generation energy efficacy, by maintaining an actual NA (numerical aperture) of the laser beam at the fiber distal tip within a range 0.02-0.08, regardless of the design NA of the fiber, even if such design NA is higher, for example, 0.1 or 0.22.

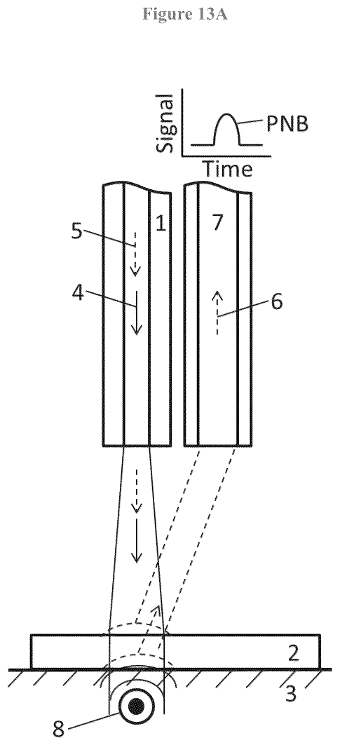

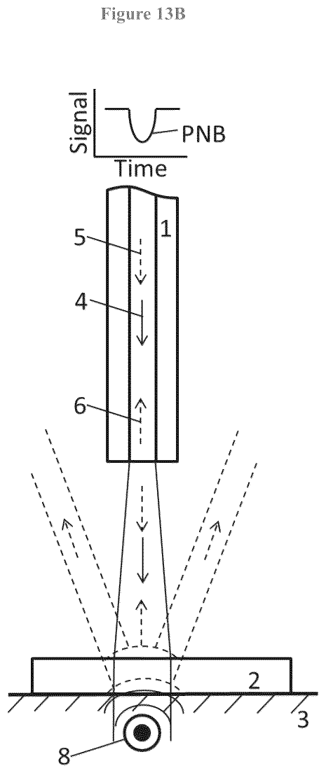

In an embodiment with the delivery optical fiber being a hollow core fiber, the pump and probe laser beams exit the fiber into the lens element where they diverge to achieve the required diameter at the tissue surface, for example, 40-50 um, or 100 um. In FIG. 13, such hollow core fiber (1) is in the center of micro-probe, and is surrounded by standard solid optical fibers (3). The pump and probe beams (solid and dashed arrows, respectively) are delivered to tissue (5) to form specific diameter, for example 50 um, through the delivery fiber (1) and the lens (4), separated by specific gap h, which allows these beam to reach for the required diameter at the tissue (5). The PNB-scattered light is collected by the lens (4) and directed through the air inside a micro-probe into collecting optical fibers (3). Collecting fibers (3) may be mounted as tight as possible around the delivery fiber (1) to minimize the outer diameter of the micro-probe and to maximize optical collection of the PNB-scattered probe laser light. The optical parameters of the lens (4), such as the focal length, shape, numerical aperture, are matched to the optical parameters of the collecting fibers (3) such as the core diameter, numerical aperture and the position, to maximize the amount of the collected and delivered through these fibers probe laser light back-scattered by the PNB(s).

The wavelength of the pump laser beam can be, for example, close to 780 nm, and the wavelength of the probe beam can be close to 1550 nm, with optical fiber, delivery and collection, optimized for these wavelengths

In any of the foregoing embodiments, to better access specific anatomic locations, the lens element of the probe can be tilted at specific angle, for example, 45 or 90 degrees, with the pump and probe laser beams delivered to the tissue surface and the PNB-backscattered probe laser light collected from there at that angle. In this case, an additional mirror or a prism can be employed between the optical fibers 1, 3 and the lens 4 to re-direct optical beams. The focal distance of the lens, in this case, may be longer, in order to accommodate additional optical element between the lens and the tissue, a prism, for example.

Flexible micro-probe provides for delivery of a probe laser beam to the tissue surface. In an embodiment, the flexible micro-probe co-delivers the probe and pump laser beams through the same optical fiber(s), using the options described above in FIGS. 6-8, 11, so a probe beam illuminates the area exposed to the pump laser beam. In another embodiment, the flexible micro-probe delivers the probe and pump laser beams through separate fibers, and spatially mixes the pump and probe beams at the tissue surface (as in FIGS. 7-8). This is achieved with one or several additional optical fibers (single mode or multi-mode), positioned around the pump delivery fiber, and directing the probe beam(s), after they exit optical fiber, into same optical element that partially focuses the pump laser beam. The mutual location of optical fiber (4) that delivers a probe beam and a lens (6) provides for the partial focusing of the probe beam(s) to the footprint of the pump beam at the outer surface of the lens and/or in the tissue surface below the lens (FIG. 7-8). For example, fiber (4) with the tip located around the tip of the pump laser fiber, and within the optical aperture of the focusing lens on the fiber side of the lens.

In an embodiment the optical element comprises a convex lens separated by air from the probe laser collecting fiber tip (5) (s), and (FIG. 7). The lens directs probe beam (s) to the tissue surface of the lens where such probe beams illuminate the tissue exposed to the pump laser beam. This can be same lens that delivers a pump laser beam to the tissue.

In another embodiment the optical element comprises an optical lens between the probe beam fiber(s) and the tissue is a grin lens (FIG. 8) which directs probe beam(s) to the tissue surface of the lens where such probe beams illuminate the tissue exposed to the pump laser beam. This can be a grin lens with a pitch between 0.2 and 0.24, and the diameter between 0.5 mm and 2.0 mm.

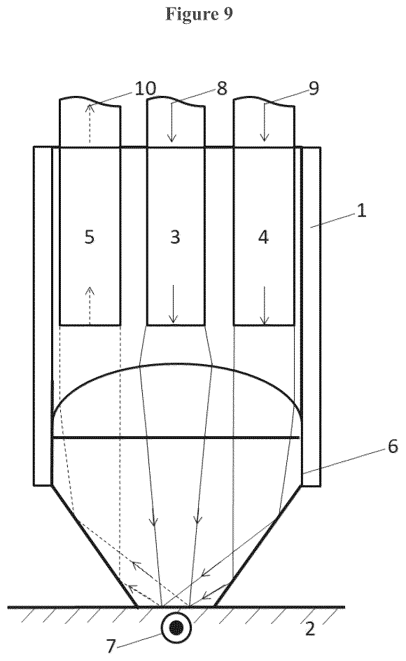

In another embodiment depicted in FIG. 9, the optical element (6) between the probe beam optical fiber(s) (4) and the tissue (2), provides illumination of the tissue side of the lens by probe laser beam at the angle, resulting in a total internal reflection of the probe beams inside such lens if the media outside the tissue lens surface becomes in air or a vapor, but does not result in a total internal reflection if the media outside the tissue surface of the lens is a condensed matter, for example, a wet soft tissue. Such lens can include an additional facet between the tissue surface of the lens and its side wall: this facet directs probe laser beams at the tissue surface of the lens at specific angle as described above. When PNB(s) is/are generated near the tissue surface of such lens they temporarily create a vapor at the outer surface of that lens. This results in a total internal reflection of the probe laser beam(s) in this location, and thus more probe laser light is directed back into the lens, and to the photodetector, changing the photodetor's signal output and thus reporting a PNB.

In an embodiment, the optical path for the probe beam is different optical path from the pump beam. In this embodiment a focusing lens focuses the probe beam(s) into the tissue area of PNB generation (FIGS. 7-9), the peripheral side of the fiber surface of the lens (6) will be modified, to direct any peripheral probe beam(s) to the center of the lens (6) and so to better illuminate the rea of PNB generation in the tissue.

In an embodiment, the probe laser beam can be co-delivered together with a pump laser pulse through the same optical fiber (3) (FIG. 6), and all peripheral fibers 4 are all used to maximize the collection of PNB-scattered probe laser light.

Collection and flexible delivery of the PNB-scattered probe laser light employs one or several optical fibers, positioned around the delivery fiber. The PNB-backscattered probe laser light is first collected by a lens (6) in the way that the maximal amount of such PNB-backscattered light is launched into collection optical fibers which deliver that light to the photodetector. In an embodiment, the lens is the same optical element that delivers the pump beam to the tissue. The mutual location of optical fibers and a lens provides for efficient optical coupling of the probe laser light backscattered by PNB(s) at the tissue surface and within the aperture of the pump laser beam, into the collecting optical fibers. Such fibers may have a high NA, for example, from 0.22 to 0.6, and a core diameter, for example, from 100 um to 600 um, with their tips located around the tip of the delivery fiber, and within the optical aperture of the lens on the fiber side of the lens. In an embodiment, the optical element is a convex lens separated by air from the collecting fiber tip(s) (FIGS. 6-9, 11). The lens collects the probe laser light, backscattered by PNB(s) in the tissue near the lens surface, and directs the collected probe light into one or several light-collecting optical fibers. This can be same lens that delivers a pump laser beam to the tissue. The outer side of the lens (its tissue side) is in optical contact with the tissue surface. In another embodiment, the optical element is an optical lens between the collecting fiber(s) and the tissue is a grin lens (FIG. 8). This grin lens collects the probe laser light, scattered by PNB(s) in the tissue near the lens tissue surface, and directs the collected probe light into one or several light-collecting optical fibers. This can be same lens that delivers a pump laser beam to the tissue. In another embodiment, the optical element is between the fiber(s) and the tissue, that collects PNB-backscattered probe laser light, has the optical geometry that results in a total internal reflection of the probe beams inside such element where the PNB is generated in a tissue surface adjacent to that element (FIG. 9). Such geometry may include a facet between the tissue surface and a side wall, at specific angle, which supports the condition of the total internal reflection when PNB(s) is/are generated near the tissue surface of such element. For the condensed matter state of a tissue (liquid and solid), the tissue surface of that optical element transmits the probe light from the element and into the tissue (FIG. 9). PNB generation near the tissue surface of such lens temporarily creates a vapor at that lens surface. This results in a total internal reflection of the probe laser beam(s) inside the optical element, in this tissue location, and thus PNB generation increases the amount of light directed into collecting optical fibers and to the photodetector. Such lens can include, for example, an additional facet between the tissue surface of the lens and its side wall: this facet directs probe laser light from peripheral fibers and onto the tissue side of the element at the angle which allows for the probe laser light into the tissue at the absence of PNB but reflects all probe light back into the element in the areas where PNB(s) is (are) generated in the tissue. This optical element can be, for example, the same lens as the one used for the delivery of the pump and probe laser beams.

In an embodiment, confirmation that the probe is in optical contact with the target tissue will use the base level of the probe laser light. This base level will be the lowest with the probe in air or water, and the highest when the focal point of the collecting lens coincides with the tissue surface (due to the increased optical scattering by the tissue). Therefore, monitoring the base level signal as function of the position of the probe would provide the confirmation of the optical contact for the probe. Additionally, the probe can be moved further by 20-50 um.

In any of the foregoing embodiments, the system comprises a pump laser and a probe laser, with the diameters of their beams on the tissue and other parameters being critical for cancer (or other target) cell detection and destruction with PNBs. Below these key parameters are considered in detail.

The probe laser. Assuming a PNB diameter Dpnb can reach up to 1 um, the optical scattering cross-section can be estimated as ¾Dpnb² and would be close to 1 um². The optical cross-section of diagnostic events will be proportional to the number N of the generated PNBs which can be approximated by the number of cancer cells: ¾Dpnb²×N um². The cross-section of the background signal can be estimated by the area of the footprint of the probe beam, ¾D²-¾Dpnb²×N. In case D>20 um the background optical scattering cross-section can be approximated by ¾D². For the integral time-response the background scattering determines the baseline signal:

Sb=Kb ¾D²

and the PNB scattering determines a diagnostic signal:

Sd=KpnbN¾Dpnb²

where Kb and Kpnb are optical scattering coefficients for the tissue and for PNB, respectively. Generally, Kpnb>>Kb, for example Kpnb=10 Kb, and the maximal diameter Dpnb of the PNB is determined by the fluence of the pump laser pulse Fp as Dpnb=A Fp, where A is a constant determined by the nanoparticle properties and by the nanoparticle binding to cancer cells. Therefore, the ratio Sd/Sb of the diagnostic signal to background signal, for integral time-response, would be:

Sd/Sb=[Kpnb/Kb][Dpnb²/D²]N

This parameter should exceed the amplitude noise of the baseline by at least factor of 2, in order to detect N cancer cells. For an ultimate detection of a single cancer cell (N=1) and assuming Kpnb/Kb=10, the typical noise level 0.01, D=20 um, Dpnb=1 um, the ratio above would be 0.025, vs the noise 0.01, and thus it will be possible to detect a single cancer cell in a tissue. However, the diameter of the probe beam in this case is close to the size of a cancer cell.

Practical considerations would suggest increasing the diameters of the pump and probe laser beams to a larger surface of the tissue to detect N PNBs instead of one PNB and thus to increase the diagnostic sensitivity. While this will increase the signal by the factor of N the background signal will increase proportionally to the square of the diameter of the probe beam, and the ratio of the diagnostic signal to the background would decrease. The present disclosure presents the following solutions, to be used alone or in combination, to increase signal for a larger probe beam diameter: (1) increase optical scattering by a single PNB by making that PNB larger through the higher fluence of the pump laser pulse (by the factor of 2-5); (2) increase the ratio Kpnb/Kb (for example, from 10 to 100, i.e. by the factor of 10); and/or (3) reduce the amplitude noise of the probe laser (by the factor of 10, from 0.01 to 0.001 or to 0.1%). These steps potentially increase the ratio of Sd/Sb up by additional factor of 500 (5×10×10) and thus will compensate a similar increase in the probe beam diameter by 20 times (a 400-fold decrease in signal to background ratio). This would make a probe beam diameter of 200 um quite realistic to detect a single cancer cell through a PNB in the background of tissue-associated optical scattering. In sum, small probe laser beams provide the highest diagnostic sensitivity and the lowest level of energy of pump laser pulse.

Increasing the diameter of the probe laser beam increases the tissue area probed by the method, providing that the diameter of the pump beam is increased proportionally. For the tissue of finite size being scanned by the laser beams larger beam diameters reduce the time to result. However, this time depends also upon the repetition rate. With 1 MHz rates available, it may be easier to scan the tissue fast enough with a small beam. For example, 20 um probe beam scan rate would be 3 cm²/s (at the linear scan speed of 20 m/s), sufficient for a real-time diagnostics of surgical bed. However, 1 MHz digitation rate for a signal may be too difficult. In case the limit is with the signal digitation at the level 1 KHz, the scan speed would be, for example: 0.003 cm²/s (7 min for 3 cm² at 1 KHz scan, scan speed 20 mm/s) for a 20 um beam and 0.3 cm²/s (10 s for 3 cm², at 1 KHz scan, scan speed 200 mm/s) for 200 um beam.

In an embodiment, a PNB signal is presented on a map image of the target tissue. This map can be obtained through scanning of laser beam (inside a rigid probe or with a rigid or micro probes). The probe, a rigid endoscope or flexible micro-probe, can be scanned across the tissue surface with a robotic device, surgical or flexible, while using the base level of the background signal to monitor the probe being in an optical contact with the tissue. The signal parameters obtained from a time-response during the scan can be mapped as X-Y image or multiple images, one image for each signal parameter. This creates an analog of digital diagnostic scanning microscope.

In an embodiment, PNB are detected as the scattering image with pulsed probe laser. This solution allows to use larger pump and probe beam diameters, and the Sd/Sb ratio will be determined by different factors: the background will be determined by optical scattering non-uniformity of the tissue which can be quite high; and the signal will be determined by the brightness of PNB which is determined by the number of clustered around cancer cells plasmonic nanoparticles and by the fluence of the laser pulse. This solution employs a fast image sensor and analyzer, with an ability to capture, feed and analyze more than 1000 frames per second, and a higher energy of the pump laser pulse. A pulsed probe laser would also increase the complexity of the design compared to a low noise c.w. probe laser.

The pump laser and delivery of a pump laser pulse. To generate cancer associated PNBs at tissue surface, single laser pulses of the duration/minimal fluence combinations at 1030-1070 nm, should be considered for the laser beam diameter 50-200 um. For a 200 um diameter of the pump laser beam at the tissue surface the other parameters would depend upon pulse length:

Minimal pump pulse energy, uJ/and an average
power, /mW, for the maximal repetition rate of 1 kHz

| Pump pulse duration, ps | 10 | 30 | 60 |
|---|---|---|---|
| Minimal pump pulse fluence, mJ/cm2 | 50 | 150 | 300 |
| Small tissue area, 20 um spot | 0.15/0.15 | 0.45/0.45 | 0.9/0.9 |
| (microscopy mode) | | | |
| Large tissue area, 200 um spot (scan | 15/15 | 45/45 | 90/90 |
| mode) | | | |

Delivery of the pulse through a flexible fiber without a laser-induced fiber damage requires to meet two requirements: (1) an optical fiber that withstands the maximal optical intensity of a picosecond pump laser pulse; and (2) coupling of a free space pump laser beam into optical fiber to prevent the laser-induced damage to the fiber or its surface. These requirements are met through the two design options: modification of the solid fused silica optical fiber or by using an optical fiber with a hollow core designed for the delivery of ultra-short laser pulse at specific wavelengths, for example, anti-resonant hollow core fiber or a hollow core photonic crystal fiber or a Cagome type hollow core fiber.

In an embodiment, to withstand maximal optical intensity of a picosecond pump laser pulse, a multi-mode step index fused silica core fiber with the core diameter from 100 um to 400 um. In another embodiment, a hollow core photonic crystal fiber with a core diameter of 10 um to 100 um um or larger is used. In another embodiment, a bundle of several fibers of the foregoing embodiments is used.

In an embodiment, coupling of a free space pump laser beam into optical fiber is achieved by any one or combination of: (i) reducing below 2 GW/cm2 a peak intensity of the pump laser pulse; (ii) directing the diverging laser beam into the fiber, so the laser beam enters the optical fiber beyond the focal point of the laser beam; (iii) reducing the beam NA (numerical aperture) below 0.07; (iv) providing the maximal diameter of the laser beam at the fiber, within 70-80% of the core diameter of the fiber; (v) using the polished and dust-free tip of the fiber; (vi) sing a fiber cap, optically fused with the fiber, with the input core diameter 2-4 times higher than the core diameter of the fiber, so to allow additional expansion of the pump beam and the reduction of its intensity; (vii) using a special optical coating of the fiber tip surface, designed to increase the air-to-fiber coupling; and/or (viii) enclosing the fiber coupling optics, fiber and unit into a sealed chamber, filled with a clean dust-free air or clean inert gas, so the surface of the fiber tip is isolated from the room air and thus is protected from an incidental dust or other damage-generating artifacts. In a preferred embodiment, for the pump pulse repetition rate up to 1 KHz and 200 um core fiber, the combination of the preceding results in an average power in mW range.

A pump pulse may have the following specifications:

Pulsed laser specs, free space

| Parameter | Unit | Value | Note |
|---|---|---|---|
| Wavelength | nm | 1000-1100 or | one or several wavelengths |
| | | 770-790 | |
| Pulse duration | ps | 5-40 | Fixed or tunable |
| Pulse shape | | Smooth single | |
| | | pulse, not a | |
| | | train | |
| Pulse energy | uJ | >20@10 ps | sufficient to provide a |
| | | >100@40 ps | fluence >100 mJ/cm2 |
| Pulse energy | % | <5 | Emax-Emin/Eavg, 100 |
| stability, short | | | consecutive pulses |
| term | | | |
| Pulse repetition | Hz | 0-1000 | Single pulses or pulse |
| rate | | | repetition rate |
| Beam diameter | mm | 0.8-3.0 | fixed |
| Beam divergence | mrad | <2 | Collimated beam preferable |
| Beam quality | M² | <1.3 | Important not to have hot |
| | | | spots within the beam aperture |
| Width of spectral | nm | <3 | |
| line | | | |

Probe laser is a continuous laser with very low intensity noise in the frequency range from 0.1 to 100 MHz, for example −150 dBc/Hz e0.5 or lower. The table below shows possible specs of such probe laser

| Parameter | Unit | Value | Note |
|---|---|---|---|
| Wavelength | nm | 620-700 | Optional 1319-1550 |
| Power | mW | 1-5 | |
| Intensity noise, peak- | % | <0.1 | For a 10 us time window |
| to-peak/baselevel | | | RIN: |
| | | | −130 dBc @ 1 MHz |
| | | | −140 dBc @ 10 MHz |
| | | | −150 dBc @ 100 MHz |
| Power stability, short | % | <2 | 10 min |
| term | | | |
| | | | This may be a fiber-coupled |
| | | | laser or a free-space laser |
| Beam diameter | mm | 2-5 | |
| Beam divergence | mrad | 0.5 | |
| Beam quality | M² | <1.5 | |
| Width of spectral line | nm | <10 | |
| Power on/off and level | | | Via computer |
| control | | | |
| Design | | | Compact rugged unit |

In an embodiment, a photodetector is a low-noise detector with a high dynamic range. Such detector, in combination with a low intensity noise probe laser, can detect transient non-stationary deviations (created by optical scattering of the probe laser beam by PNB(s) in tissue) from a base level stationary signal (created by optical scattering of a probe laser beam in tissue) as small as 0.001-0.0001 of the level of the base signal, and in the signal frequency range 0.1-100 MHz. For example, this is a NIR amplified or/and balanced (or auto-balanced) detector which may use an additional reference optical channel to receive and subtract the signal produced by the laser alone or by a tissue in the area where no PNBs can be generated. Below is an example of the detector specs:

| | | Detector type | |
|---|---|---|---|
| | | FPD 510 NIR or | PDB570C or |
| | | similar mono | similar balanced |
| Parameter | Unit | detector | detector |
| Wavelength | nm | 1550 | 1550 |
| Diameter of the | mm | <0.3 or s fiber- | Single mode fiber |
| optical element | | coupled port, | coupled ports, 2 |
| | | FC/APC | FC/APC |
| Bandwidth | MHz | DC - 200 | DC-400 |
| Noise (NEP) | pW/vHz | 3 | 1 |
| Number of inputs | | 1 | 2 (signal and |
| | | | reference) |
| Gain | V/W | 5 × 10e4 | 1.8 × 10e4 |
| Noise subtraction | | n/a | 30 dB, fixed and auto |

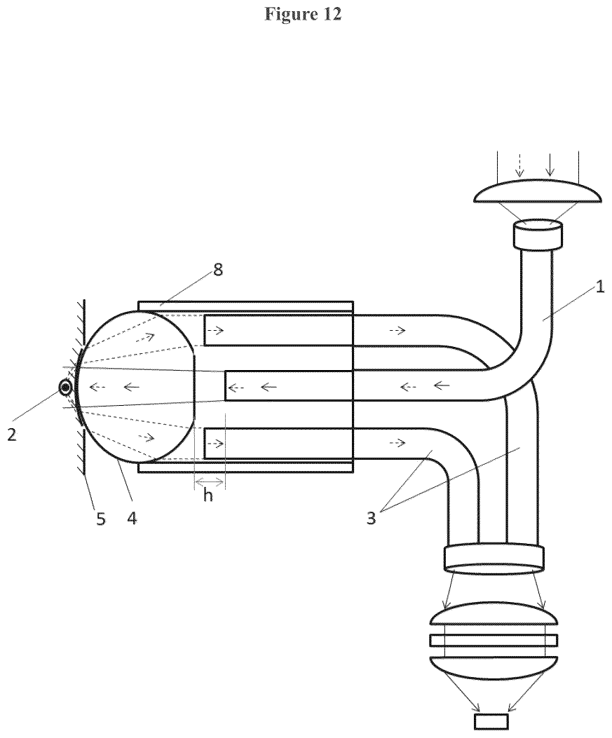

In an embodiment, shown in FIG. 12, where a flexible endoscopic micro-probe connected to a laser base unit with a flexible optical fiber bundle, bifurcated on its proximal end, into two legs, each connected to a fiber coupling unit in the base laser unit: the delivery hollow core fiber 1 is coupled to a pump and probe laser beams, the collection optical fibers 3 are optically coupled to the photodetector which receives a PNB (2)—backscattered probe laser light through the collection fibers 3. Distal ends of delivery fiber 1 and collection fibers 3 are bundled into a casing 8 and deliver the probe and pump laser beams to the tissue 5 through the lens 4 which has a flat surface in its back and is separated from the tip of the delivery fiber 1 by the gap h, and directs the pump and probe laser beams into the tissue 5, and collects the probe laser light back-scattered by PNB 2 and direct such light into collection fibers 3 The photodetector may have a free space or an optical fiber-coupled photosensitive element

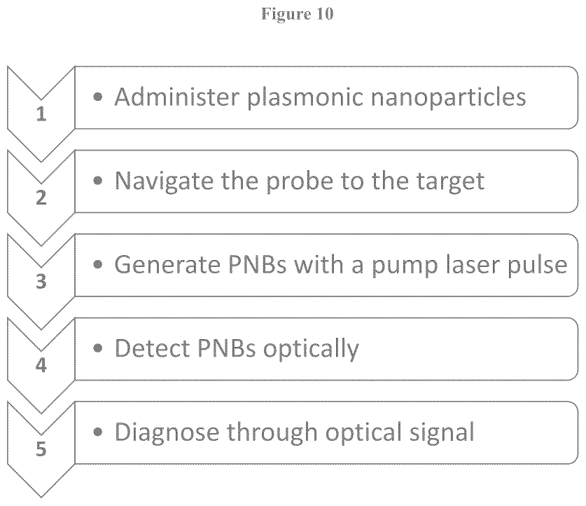

The method disclosed herein comprises: (1) administering to a patient plasmonic nanoparticles; (2) navigating the probe disclosed herein to the target tissue; (3) generating PNBs with a pump laser pulse; (4) detecting PNBs optically through the backscattering of a probe laser light by a PNB; and (5) diagnosing the target tissue (see FIG. 10) by analysis the optical signal collected from the tissue.

In a preferred embodiment the method further comprises the step of (6) treating the target tissue based on the results of the diagnosing step, by using PNBs or other treatment method.

In an embodiment, the plasmonic nanoparticles are administered locally to the target tissue. In another embodiment, the plasmonic nanoparticles are administered systemically. In any of the foregoing embodiments, the plasmonic nanoparticles comprise a plasmon resonance wavelength similar to that of the pump laser pulse, in a preferred embodiment in the range of 600-1100 nm. In an embodiment, the plasmonic nanoparticles comprise hollow gold nanoparticles coated with PEG. In an embodiment, the plasmonic nanoparticles comprise a surface coating that targets them to cancer cells, for example, a monoclonal antibody to EGFR, which is known to be up-regulated in cancer cells. In an embodiment, the nanoparticle metal concentration comprises 2-4 mg/kg of body weight.

In an embodiment, the wavelength of the most efficient PNB generation may be in NIR spectral window, for example, at 782 nm, and differs from the spectral peak of optical stationary plasmon resonance of plasmonic nanoparticles, which can be, for example, in the range 550-650 nm. The PNB generation with a short picosecond pulse employs the mechanism of self-inducing a transient plasmonic structure at the surface of the light-converting plasmonic nanoparticles, that transient structure efficiently absorbing a shot picosecond optical pulse at the pump laser wavelength and thus generating a PNB

In an embodiment, two or more different types of nanoparticles are administered to target cancer cells with different molecular targets, by using more than one targeting vector, for example, several different conjugated antibodies that target different cancer-specific molecular targets. In this embodiment, two or more different types of nanoparticles are used, of different plasmonic properties, to respond to the optical pumping with laser pulses at different wavelengths.

The pump laser pulse is delivered to a target tissue to generate PNBs around plasmonic nanoparticles in cancer cells. In an embodiment, the pump laser pulse is a short picosecond pulse in the range 5-40 ps, with a small footprint on a tissue, 10-200 um in diameter, and a fluence above the PNB generation threshold, in the range 10-150 mJ/cm2, at the wavelength that matches the maximal energy conversion through a plasmonic mechanism in nanoparticles, such mechanism can be permanent or transient.

In another embodiment, the fluence and the diameter of the pump laser pulse at the tissue are optimized to detect and treat cancer in a thin surface layer, so not to damage underlaying critical organs and structures, for example the beam diameter of 30 um and fluence of 50 mJ/cm2 to generate PNBs within a 100 um thick surface layer of the tissue.

In an embodiment, several simultaneous pump laser pulses are applied at different wavelengths, to pump different nanoparticles associated with such wavelengths.

In an embodiment, a pump laser pulse is delivered to a target tissue via the flexible micro-probe, with rigid or optical fiber flexible end-pieces, small and flexible enough to reach for the target tissue in a minimally invasive way, with or without a surgical procedure. In an embodiment, a pump laser pulse is scanned within the aperture of a rigid endoscope, to probe different locations in a tissue. In some embodiments, more than one consecutive pump laser pulses are applied to same location of a tissue.

The step of detecting PNBs in the target tissue comprises delivery of a probe laser beam to the tissue surface and detection of backscattering of probe laser light by PNBs if they are present. In an embodiment, the probe laser is a continuous laser with a very low relative intensity noise withing the frequency and time domains associated with the PNB signal, for example, in the time-window from 1 us to 100 us, and a frequency range from 0.01 MHz to 100 MHz. In an embodiment, the probe laser has a wavelength which is not absorbed by tissues, and is different from a pump pulse wavelength, for example, in the range 590-700 nm, or at 1500-1600 nm while the pump laser wavelength his, for example, close to 780 nm. In an embodiment, the probe laser has a power sufficient to provide enough backscattered by a PNB light for the PNB detection by a photodetector, like in the range 0.1-10 mW, for example, but low enough to avoid any thermal damage to the tissue. In an embodiment, the probe laser beam is a pulsed beam of the duration from 1 ns to 10 ns, and time-delayed relative to the pump pulse by 20-100 ns, in order to image PNBs. In an embodiment, the probe laser beam is delivered to a tissue through a flexible optical fiber endoscope probe. In an embodiment, the probe beam if focused to a small enough spot, for example, 40 um, to detect PNBs in single cancer cells with high sensitivity and specificity. In an embodiment, the probe beam is delivered to the tissue in the way that a PNB creates a total internal reflection of the probe beam in the probe and thus improves the sensitivity of the PNB detection. In an embodiment, the probe beam is delivered to the target tissue and is collected from the target tissue via flexible optical fibers, to allow minimally invasive access to restricted target tissue. In an embodiment, the probe beam is delivered to and collected from a surgical bed to probe the surface of a surgical margin where cancer cells would generate PNBs. In an embodiment, the probe beam is co-delivered with the pump beam to the side of the probe.

In an embodiment, the step of detecting PNBs in the target tissue further comprising an optical element (FIG. 13) between the tissue and optical guides capable of being optically transparent for a pump laser beam, wherein a front or back surface of the optical element is capable of internally scattering the probe laser light back into the collection optical guide (FIG. 13A), wherein the tissue changes the optical properties of the optical element at the probe laser beam wavelength, for example, it flexes, moves or vibrates when plasmonic nanobubble is generated near that surface, and wherein plasmonic nanobubble-induced changes of that surface influence the intensity, power or phase or frequency or polarization of probe laser light that was reflected from or scattered by that surface and was delivered through the collection guide to the photodetector, said influence resulting in plasmonic nanobubble-specific changes of the photodetector output signal. In FIG. 13, A delivery guide (1) delivers pump (4) and probe (5) laser beams to the tissue (3) through a flexible optical element (2), such optical element is designed to deliver the pump laser beam (4) from the fiber guide (1) to tissue (3), to reflect or scatter the probe laser beam (5) by its back or front surface, and to flex in response to the generation of plasmonic nanobubble (8) so that the portion of the reflected or scattered probe laser light (6) is directed to the collection guide (7) (A) so the probe laser light delivered by the collection optical guide (7) is caused by a PNB 8 which, in turn, causes the flexing of the optical element 2. Figure A shows a PNB-induced increase in the photodetector signal, relative to its base level in the absence of a PNB, due to the increase of the scattered/reflected probe laser light (6) through the optical collection guide (7). FIG. 13B shows a PNB-induced decrease in the photodetector signal, relative to its base level in the absence of a PNB, as detected the scattered/reflected probe laser light (6) through the delivery guide (1). In this case, the PNB 8—backscattered light 6 is collected and delivered to the photodetector by the delivery optical fiber 1

In an embodiment, the method further comprises analysis of photodetector signals to determine a PNB-positive signal. This analysis comprises any one or combination of the following parameters: (i) the signal shape, amplitude and duration, time-position of the signal, the level of the baseline, the level of the background (measured prior to the exposure of the tissue to the pump lase pulse); (ii) the lateral position of the pump laser pulse at the tissue surface: at the distal end of a rigid endoscope or the position of the flexible micro-probe; (iii) the pump pulse energy (fluence): For the surface-only diagnostics of the tissue in the depth range from 0 to 50 um, apply the minimal pump pulse fluence, for example, in the range 60 to 80 mJ/cm2. For diagnosis deeper tissue ranges from 0 to 100 um, apply the higher fluence, for example, from 100 to 140 mJ/cm2. In an embodiment, based on the PNB status of the signal, the position and fluence of the pump laser pulse the algorithm will conclude whether the signal is cancer-positive or negative. Such algorithm will use a set of user-defined thresholds to compare the actual parameters against those thresholds. In another embodiment, a diagnostic algorithm is designed to determine a cancer status of the tissue location within microseconds to milliseconds after a pump laser pulse, so to support the next, treatment decision.

In an embodiment, the method further comprises guided destruction of target cells that were detected by the photodetector through a PNB signal. The guided destruction of target cells may be manual, for example where an operator decides on the treatment, a surgical or else. This is so called PNB-guided surgery or other cancer treatment applied by a human operator. In another embodiment, the guided destruction of target cells is automated, wherein an algorithm automatically administers PNB treatment, in case of a positive diagnosis, by sending additional 1-20 laser pulses of the increased fluence to the same location in the tissue and through the same probe. In this case, the follow-up consecutive PNBs in the same location mechanically damage or destroy cancer cells there. In this, automatic theranostic case, an algorithm can also use optical signals of treatment PNBs to monitor the treatment process in a real time. In another embodiment wherein the guided destruction of target cells is automated, an algorithm automatically uses non-PNB treatment options supported by another device controlled through this algorithm, for example, a surgical robot, to apply in real time a device-specific treatment to a cancer- or other disease-positive location determined through PNB diagnostic signals.

In an embodiment, the method further comprises a step of chemotherapy and gene and cell therapy treatment with the endoscope or biopsy needle. In a preferred embodiment, the method comprises: Step 1. Administer gold nanoparticles in vivo into, for example, the blood flow, conjugated with cancer-specific monoclonal antibodies, to target cancer cells or a tumor. Co-infuse or co-inject into the blood flow liposomal form of the drug or genetic cargo, conjugated to the same monoclonal antibody. Within 24 hours, drug liposomes and gold nanoparticles will accumulate at the tumor; Step 2. In 24 hours (or at other specific time interval), bring the PNB micro-probe in contact with the surface of the target tissue or organ. Administer pump laser pulses at specific optical fluence to generate small PNBs around gold nanoparticles. Monitor the PNB generation optically. These relatively small PNBs may not mechanically destroy the tumor (or other target cells) but they will release the drug from liposomes (or other carrier disrupted by PNBs) thus providing a high local concentration of the drug inside cancer cells or inside the tumor. At the same time, systemic concentration of the released drug may remain very low because the PNB-activated drug release will take place only in the limited area exposed to a pump laser beam.

In an embodiment, the method further comprises a step of gene therapy treatment with the endoscope or biopsy needle or catheter. In a preferred embodiment, the method comprises: Step 1. Administer gold nanoparticles in vivo into the blood flow, conjugated with anti-CD3 antibodies, to target CD3-positive lymphocytes. Co-infuse or co-inject into the blood flow genetic material, encapsulated into liposomes or similar carrier, conjugated to the same monoclonal antibody. Within 24 hours, plasmid liposomes and gold nanoparticles will accumulate at CD3-positive lymphocytes in blood; Step 2. In 24 hours, insert the PNB micro-probe into a blood vessel. Administer pump laser pulses at specific optical fluence to generate small PNBs in CD3-positive lymphocytes passing close to the output window of the micro-probe. Monitor the PNB generation optically. These PNBs will release genetic cargo from liposomes and will deliver such genetic cargo (for example, plasmid) into the cytoplasm of CD3-positive cells, to ensure their genetic transfection and thus converting these cells into natural killer cells.

In an embodiment, the method further comprises confirmation of optical contact between the probe and target tissue, using use the base level of the photodetector output signal which characterizes the backscattered probe laser light: this level will be the lowest with the probe in air or water, and the highest when the focal point of the collecting lens coincides with the tissue surface (due to the increased optical scattering by the tissue). Therefore, monitoring the baselevel signal as function of the position of the probe would provide the confirmation of the optical contact for the probe.

In another embodiment, A probe in the distal end has the minimal diameter possible (0.5-3 mm) and a short length, 3-30 mm, is connected to the fiber bundle, and supports three optical functions while being in optical contact with target tissue: (1, 2): Delivers the pump and probe laser beams into the target tissue surface, with specific diameter of the laser beams at tissue-probe interface, for example, 50 um, the pump and probe beam should overlap and coincide at the interface surface; and (3): Collects of PNB-backscattered probe light from the tissue surface so that the maximal amount of the backscattered light is delivered through the fiber bundle to the photodetector. The source of backscattered light, one or several PNBs, can be located anywhere within the volume illuminated by the pump laser beam in the depth range (from tissue-probe interface) from 0 to 50 um.

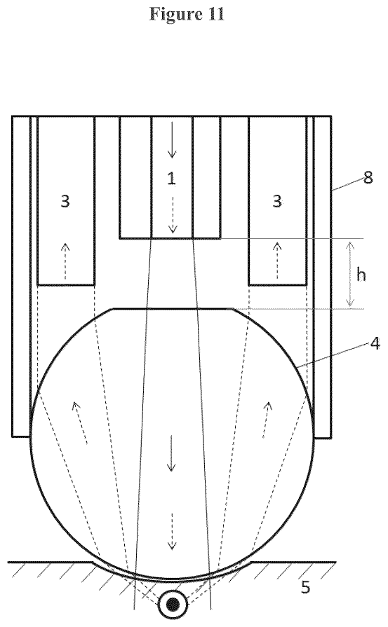

This probe may use a co-delivered pump and probe laser beams as shown in FIG. 11. Note these functions require different management of outcoming and incoming optical beams in the lens (4).

For collecting the backscattered light, an optical focus [of the probe] should be close to the surface of the lens, 0-100 um away from it.

For the delivery of probe and pump laser beams, they should not be focused at the surface, and have a specific diameter of in the range from 30 to 200 um. This requirement suggests their focal points can be quite far from the surface, or the beams may slightly diverge. The beam diameter Dbeam at the inner surface of the lens (4) is determined as:

Dbeam=Dmfd+2gap NA

where NA is the fiber NA, and gap is the distance between the lens surface and the fiber tip, Dmfd is the beam diameter at the fiber output (mode field diameter).

The beam diameter at the tissue/lens interface can be precisely adjusted for any specific hollow core fiber through the variation of the gap between the fiber tip and the back surface of the lens. This axial adjustment of the position of the fiber tip allows for using the most appropriate hollow core fiber. For typical hollow core fiber NA=0.02, the beam diameter, in um, would be Dmfd+0.04 gap, each millimeter of the gap adding 40 um to the beam diameter.

PNB probe design and requirements as shown in FIG. 11: (a) The diameter of the pump beam (solid arrow) at the probe-tissue surface interface: from 30 um to 200 um, (optional 50 um); (b) The pump beam is delivered with Hollow Core Fiber (HCF) (1), located in the center of the micro-probe and in front of the flat of the ball lens (4); (c) The delivery fiber (1) is inserted through an internal guide, a metal tubing. There is an air gap h between the distal tip of the hollow fiber (1) and the flat surface of the ball lens (4) (the gap). This gap may be adjusted in the range 0-2 mm so to achieve the specified diameter of the beam at the lens-tissue interface; (d) The pump beam and probe beam are not focused at the tissue to their focal points. Their propagation in the lens is rather determined by out-of-fiber NA (0.02-0.05), to form the beam diameter 50 um (or optional 100 um) at the lens bottom surface. This may be achieved with a flat back surface of the ball lens (4); (e) The internal metal tubing acts as a guide for a delivery fiber: it allows moving the fiber, supports it and protects it in the back. Both edges of the tubing must be round and dust-free; (f) The probe beam (dashed arrow) is co-delivered together with the pump laser beam, through the same delivery fiber to spatially coincide with the footprint of the pump beam at the probe-tissue surface interface. The diameter of the probe beam at the interface should be close to that of the pump beam; (g) The collecting optical fiber or fibers (3) is silica step index fiber with NA 0.22 and core 200 um. The number of collecting fibers (3) is 7, can be from 4 to 100, to increase the collection of backscattered by PNB light. An optional design is to use 7 peripheral fibers, packed around the delivery fiber; (h) The lens (4) has to match additional requirements: (i) efficient collection of the backscattered probe laser light (1550 nm) from point sources located in the tissue or liquid media and close to its surface, 10-40 um from the lens. This suggests a ball lens geometry with the material of the lens such as sapphire having a high refractive index in order to efficiently collect the light and to collimate it into the collection fibers (ii) the surface of the lens should be as hard as possible, in order to avoid being damaged by the mechanical impact of exploding PNBs close to the lens surface. A sapphire material would match that requirement. (iii) Option: the lens should have AR coating for the probe laser wavelength in order to maximize the collection of the probe light from a PNB (iv) to collect and direct as much as possible light to collection fibers, in peripheral area, a lens shape of a ball type may be preferred (v) possible shape of a ball lens: a sphere or a sphere with a flat (diameter in the range 0.4-0.5 mm, FIG. 11). A flat section in the back of the lens matches the footprint of the delivery fiber and prevents excessive focusing of the pump and probe beams in the tissue.

In another embodiment, the correct placement of the probe in the tissue, with the proper optical contact with the tissue, is achieved with three possible mechanisms:

Mechanism 1: optical interference of the probe beam reflected from the back of the lens (c.w. 1550 nm)

The design of Mechanism 1 is as follows: Reference beam R: from the laser-through 50% splitter and return prism and attenuator. Signal beam S: from the delivery or collection fibers, the part reflected back from the inner surface (flat) of the ball lens. The uncoated flat of the ball lens reflects 4% of the incident beam, in the air. The reflected beam gets back into the delivery fiber, exits the delivery fiber through the coupler FC1 and travels back to the probe laser. This can be a small fraction of the output of the probe laser: 40% (out of the fiber)×4% (reflected from the lens)×30% (reverse fiber coupling efficacy)×40% delivered to the sensor=0.2% of 2 mW=3-4 uW. Similar reference power should be used. The reflecting back surface of the ball lens should move in axial direction when its front surface is in contact with the tissue. This is achieved through elastic glue which attaches the lens to the casing. The axial movement of the lens should be within micrometers (<1 um). The lens should return into its initial position when the pressure is removed. Residual shift can be compensated with a self-calibration of the sensor.

The operation of Mechanism 1 is as follows: interference of the reference and signal beams creates the intensity distribution which is sensitive to the movement of the lens. The intensity is measured continuously with the photodetector. The electric output of the photodetector correlates to the optical contact between the probe and tissue. A photodetector output is read against two thresholds: (a) Free probe threshold 1: when the signal is less than the threshold the probe is not in contact with tissue and can be scanned in a lateral direction; (b) Probe in tissue threshold 2: when the signal is above the threshold 2 the probe is in sufficient contact with tissue to perform PNB protocol.

Mechanism 2: optical scattering of the probe beam by the tissue

The design of Mechanism 2 is as follows: unmodified probe is used with the ball lens secured to the probe casing via solid glue. The background scattering of the probe beam from the tissue is measured as function of the probe-tissue relative position: A probe not in contact with tissue creates the minimal scattering signal level 1; A probe in tissue increases the optical scattering of the probe beam, with the maximal scattering signal level 2.

The operation of Mechanism 2 is as follows: The levels 1 and 2 are determined experimentally during the probe calibration on a specific tissue.

Mechanism 3: optical reflection of the probe beam by the front surface of the ball lens depends upon optical contact with the tissue

The design of Mechanism 3 is as follows: monitor the fraction of the probe laser beam reflected from the front surface of the probe lens. It depends upon the refractive index of the outer media. The refractive index is the highest (and the reflection is the lowest) with good contact with the tissue. The refractive index decreases as the probe is lifted and loses good optical contact with the tissue, increasing the reflected light. In such a position the probe, not in optical contact with the tissue, can be moved/scanned across the tissue without dragging or stretching the tissue mechanically.

A calibration algorithm can be fully automated in support of a high-speed scan procedure: Move a probe from a non-contact position until it is relatively deep in the tissue, and record the signal as the function of the height above the tissue/depth in the tissue. There will be two signal plateaus, for the probe outside of the tissue and inside the tissue, and a gradient transition from one plateau to another. Each plateau is due to a relatively permanent optical properties of the media outside and inside the tissue, for a small footprint of the probe. The tissue surface creates a gradient of the signal due to the transition between refractive index and transparency of the media. This gradient transition marks the two positions (thresholds) of interest for the probe: one, Thr 1 in optical contact with tissue to perform PNBs, and another, Thr 2 not in contact with the tissue, to perform safe lateral movements of the probe without a risk of dragging/damaging the tissue. Two height points, corresponding to the start and end of the gradient, will be used for PNB generation/detection and for safe scanning of the probe. The algorithm can be repeated as frequently as needed to accommodate for a heterogenous tissue.

1-78. (canceled)

79. A method for diagnosing a tissue in a patient comprising: (a) administering to a patient metal nanoparticles; (b) navigating a probe to a target tissue; (c) generating plasmonic nanobubbles with a pump laser pulse delivered through the probe; (d) detecting plasmonic nanobubbles optically in vivo; and (e) diagnosing the target tissue through analysis of the detected optical signal in response to a pump laser pulse.

80. The method of claim 79, wherein the nanoparticles comprise particles capable of developing transient non-stationary plasmon resonance properties during their exposure to a pump laser pulse, such properties absent under exposure of the particles to continuous pump laser beam or a pulsed laser beam with suboptimal duration and intensity of a pump laser pulse.

81. The method of claim 79, wherein the nanoparticles comprise particles conjugated with cancer-specific molecules.

82. The method of claim 79, wherein the nanoparticles comprise two or more types of nanoparticles, wherein each type of nanoparticles comprises different plasmonic properties stationary or transient non-stationary, capable of generating plasmonic nanobubbles while exposed to two or more simultaneous pump laser pulses having different wavelengths that match plasmonic properties of said nanoparticles.

83. The method of claim 79, further comprising the step of delivering consecutive pump laser pulses to the same tissue location, thereby improving the diagnostic and/or therapeutic effect of plasmonic nanobubbles.

84. A method for optical detection of plasmonic nanobubbles in tissue comprising:
(a) illuminating a pump laser-exposed tissue with a probe laser light at a time when a pump laser pulse arrives into the tissue;
(b) collecting the probe laser light scattered by the pump-laser exposed tissue to a photodetector capable of measuring the relative temporal changes in the intensity and power of the collected probe laser light;
(c) detecting a relative change in the intensity of a probe laser light scattered by plasmonic nanobubble;
(d) identifying one or more output signal component specific for a plasmonic nanobubble.

85. The method of claim 84, wherein the one or more output signal component comprises a bell-shaped signal components with a peak, negative or positive, relative to the signal baseline.

86. A method of claim 84 for detecting target cells with plasmonic nanobubbles, comprising:
(a) exposing the cells to one or more pump laser pulses at specific wavelength, duration and fluence in the range from 20 mJ/cm2 to 150 mJ/cm2;
(b) exposing the same cells to a probe laser light at the time it receives a pump pulse;
(c) collecting and analyzing a probe laser light as an optical signal;
(d) deriving quantitative parameters from the signal within the time interval from 5 ns to 2 us after the exposure of the tissue to a pump laser pulse;
(e) comparing such quantitative parameters against pre-determined diagnostic thresholds for a target cell type; and
(f) determining the presence of the target cell type, wherein the target cell type is present if one or more parameters of the signal of the collected probe laser light match a diagnostic threshold.

87. The method of claim 84, further comprising running an algorithm that compares the quantitative parameters of the probe laser light-detected signals (detected in response to pump laser pulses) to pre-determined thresholds, wherein the comparison concludes whether the cells are disease-positive or -negative (without a human decision being involved), wherein in the case of disease-positive conclusion, the method further comprises the generation of additional pump laser pulses of the increased fluence to the same location, while the probe remains in contact with tissue, with the fluence increased to the level in the range from 100 mJ/cm2 to 250 mJ/cm2.

88. A method of claim 84 for intraoperative automated detection of residual cancer cells in a surgical cavity comprising:
(a) a probe brought in optical contact with the cavity tissue at specific location of a surgical cavity;
(b) applying a pump laser pulse and a probe laser light;
(c) collecting and analyzing the probe laser light;
(d) automatically determining cancer status of a cavity tissue in contact with the probe; and
(e) producing the diagnostic data, including the cancer status and the location of the probe.

89. A method of claim 84 for eradication of cancer cells in a target tissue comprising:
(a) detecting cancer cells in a target tissue using the method of claim 6;
(b) exposing the target tissue with a pump laser pulse while the probe remains in optical contact with the diagnosed tissue;
(c) applying one to twenty pump laser pulses at specific wavelength, duration, and fluence in the range from 70 mJ/cm2 to 250 mJ/cm2;
(d) delivering pump laser pulses to the same tissue location through the probe;
(e) monitoring the therapeutic effect of each pump laser pulse through optical detection of plasmonic nanobubbles, with quantitative parameters derived for each optical signal;
(f) comparing the signal parameters against pre-determined therapeutic thresholds; and
(g) adjusting the fluence of next pump laser pulse if the signal parameters did not match the therapeutic thresholds during the previous laser pulse.

90. A device for optical generation of plasmonic nanobubbles in tissue comprising:
(a) a probe with a flexible optical guide connected to a distal optical probe, capable of delivering a pump laser pulse, wherein the flexible optical guide is configured to deliver pump and probe laser beams from one or more sources to a probe in contact with tissue without distorting spectral, temporal and energy properties of the laser beams;
(b) an interface optical element in the probe capable of providing an exposure of the tissue volume with a pump and probe laser beams; and
(c) a tip of the flexible optical guide capable of being positioned at a distance from about 5 um to about 2 mm from a back surface of the interface optical element, wherein the combination of the distance between the tip and the back surface of the interface optical element, and thickness and refractive properties of the interface optical element forms a diameter of the pump laser beam in the range from 20 to 200 um in tissue in the tissue depth range from about 0 um to about 400 um from the probe surface, and wherein the direction of the pump laser beam in the tissue along with optical axis of the flexible optical guide in the probe is capable of generating plasmonic nanobubbles in front of the probe.

91. A device of claim 90 for optical detection of plasmonic nanobubbles in tissue [in vivo] comprising:
(a) a probe laser beam,
(b) a pump laser beam;
(c) an interface optical element, wherein the interface optical element collects probe laser light scattered or reflected by plasmonic nanobubbles generated in tissue volume exposed to laser beams; and
(d) a flexible optical guide capable of delivering the collected probe laser light to one or more remote photodetectors, wherein the one or more remote photodetectors can generate an electrical output signal specific to a plasmonic nanobubble.

92. The device of claim 90, wherein the probe laser beam and the pump laser beam are capable of being co-delivered into tissue such that both beams overlap or coincide in the tissue.

93. The device of claim 90, wherein the probe laser beam comprises a diameter, wherein the diameter is limited to minimize the background of the light scattered by the tissue.

94. The device of claim 90, further comprising an optical element in the probe capable of delivering the pump and probe laser beams from the fiber guide to the tissue without focusing them, i.e. maintaining the desired diameter D in the tissue near the probe, and to collect and collimate the probe laser light scattered by a plasmonic nanobubble generated in the tissue near the probe.

95. The device of claim 90, wherein the probe laser beam is capable of being internally reflected from or scattered by the optical interface surface between the tissue and the probe such that a scattering of the probe laser light changes when a plasmonic nanobubble is generated in the tissue close to the interface surface, and wherein said changes in the scattering of the probe laser light are capable of being optically detected as a signal associated with the plasmonic nanobubble.

96. The device of claim 90, further comprising a photodetector at the proximal end of the probe that has a single photosensitive element capable of converting a probe laser light intensity, phase, polarization, duration or wavelength into an electrical signal of a time-amplitude or space-amplitude type.

97. The device of claim 90, wherein the probe is routed through standard minimally invasive clinical tools, flexible endoscopes (or similar endo-tools like bronchoscope and endomicroscope or else), biopsy needles or catheters.

98. The device of claim 90, further comprising a compact optical probe of the diameter not to exceed 2 mm, wherein the compact optical probe is capable of optically generating and detecting plasmonic nanobubbles in tissue near the tissue-probe interface, wherein the compact optical probe comprises a free-space optical guide capable of transmitting pump and probe laser beams through a rigid guide from laser sources to the probe, and from the probe to the photodetector(s) and signal hardware and software.
